(19) United States
(12) Patent Application Publication
(10) Pub. No.: US 2026/0285847 A1
(43) Pub. Date: Sep. 24, 2026

(54) SYMMETRIC AMINOINDOLE-LINKED TRIAZINE FOR A DUAL EFFECT ON ALPHA-SYNUCLEIN AND TAU ISOFORM 2N4R FIBRILLIZATION

(71) Applicant: Purdue Research Foundation, West Lafayette, IN (US)

(72) Inventor: Jessica Sonia FORTIN, West Lafayette, IN (US)

(21) Appl. No.: 19/168,659

(22) PCT Filed: Mar. 21, 2024
(86) PCT No.: PCT/US2024/020926

§ 371 Date: Sep. 24, 2025

Related U.S. Application Data
(60) Provisional application No. 63/454,357, filed on Mar. 24, 2023.

(51) Int. Cl. C07D 403/14; A61K 31/53; C07D 403/12

(52) U.S. Cl. CPC C07D 403/14; A61K 31/53; C07D 403/12

(57) ABSTRACT

A compound of the formula: Formula (I), (I) or Formula (II), (II) wherein R₁ is 4-aminoindole, 5-aminoindole, 6-aminoindole, or 7-aminoindole; R₂ is 4-aminoindole, 5-aminoindole, 6-aminoindile, or 7-aminoindole; and X is H, halo, NO₂, NH₂, OH, or O-alkyl; and A, B, G, X, Z, R⁴ and x are defined herein; a pharmaceutical composition comprising same; and a method of inhibiting tubulin-associated unit (tau) and/or alpha-synuclein (a-syn) protein aggregation in a subject having, or at risk for, tau and/or α-syn protein aggregation comprising administering to the subject the pharmaceutical composition in an amount effective to inhibit tau protein and/or α-syn protein aggregation.

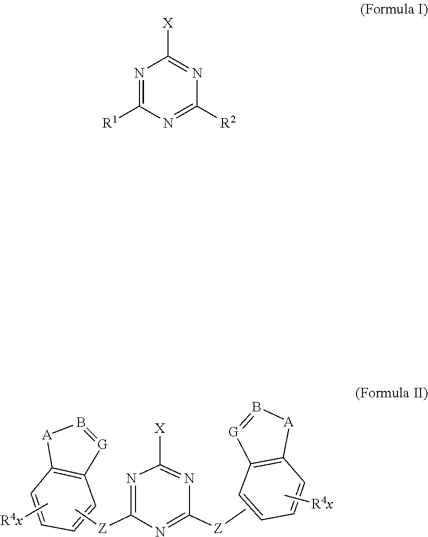

### CROSS-REFERENCE TO RELATES APPLICATIONS

This application claims the benefit of U.S. Provisional Appl. No. 63/454,357, filed Mar. 23, 2023, which is incorporated by reference as if fully set forth herein.

### STATEMENT OF GOVERNMENT SUPPORT

This invention was made with government support under NIA NIH 1 R21 AG070447-01A1 and NIA NIH 1K08AG071985-01A1 awarded by the National Institutes of Health. The government has certain rights in the invention.

### TECHNICAL FIELD

This disclosure relates to symmetric aminoindole-linked triazines, compositions comprising same, and the use of such compounds and compositions to inhibit tubulin-associated unit (tau) protein and alpha-synuclein (α-syn) protein aggregation, including, but not limited to, neurofibrillary tangles (NFTs), associated with tauopathies (e.g., Alzheimer's disease (AD), Downs syndrome, progressive supranuclear palsy, and traumatic brain injury) and Lewy bodies, associated with synucleinopathies (e.g., Parkinson's disease (PD), dementia with Lewy bodies (DLB), and multiple system atrophy (MSA)).

### BACKGROUND

The accumulation of abnormally folded protein in the brain is a common feature of progressive neurodegenerative disorders, including Alzheimer's disease (AD) and Parkinson's disease (PD). AD and PD are the most common tauopathy and synucleinopathy, respectively, characterized by the accumulation of amyloid-like fibrils composed of tau and alpha-synuclein (α-syn). Accumulating evidence suggests that, in these diseases, toxic misfolded intermediate forms of protein convert non-deleterious monomers resulting in propagation of distinct misfolded protein conformations in a stereotypical pattern across neuroanatomically connected brain regions via a prion-like mechanism.

This hypothesis has led to the pursuit of therapeutic strategies to slow down or inhibit the fibrillization. Many studies have shown that neurodegenerative disorders often exhibit comorbid pathological protein aggregation. Typical AD brains exhibit extensive accumulation of extracellular amyloid beta (Aβ) peptides in neuritic plaques as well as intracellular neurofibrillary tangles (NFTs) composed of misfolded and phosphorylated tau. However, more than half of AD cases have been characterized by the presence of Lewy bodies (LBs) enriched with misfolded alpha-synuclein (α-syn), and, in many instances, LB disorders have been reported to have concomitant Aβ plaques and NFTs. Considering the frequent co-occurrence of α-syn and tau inclusions in the brains of patients afflicted with AD and LB disorders, the discovery and development of small molecules targeting multiple pathologically relevant prone-to-aggregate proteins can be a promising potential therapeutic strategy to slow down cross-reactivity and inhibit comorbidity among misfolded proteins in numerous neurodegenerative diseases.

In the search for potential small molecule inhibitors of these deleterious misfolded neuropeptides, the triazine has garnered attention. This tridentate linker has been widely explored for the discovery and development of antifungal, anticancer, antimicrobial, and antiviral agents. Specifically, the application range of triazine-based compounds has been extended to neurodegenerative diseases, and some of these new compounds have shown multi-target properties of anti-AD or anti-PD agents. Triazine-derivatives have been found to be effective in inhibiting Aβ peptide aggregation, ameliorating Aβ peptide-induced toxicity in neuronal cells, and inhibiting the activity of BACE-1 involved in the cleavage of the β-amyloid precursor proteins. Furthermore, diphenyl triazine hybrids have been shown to inhibit α-syn fibril formation and to disaggregate preformed α-syn aggregates in vitro. However, the effects of triazine-based compounds on tau aggregation have not been completely explored. Recent insights consider tau protein as an important therapeutic target, after many Aβ peptide-targeting treatments failed in clinical trials for AD. In view of the foregoing, a series of triazine-derived compounds was synthesized, and the inhibitory effects of the compounds on tau and α-syn aggregation was examined.

### SUMMARY

Provided is a compound of the formula:

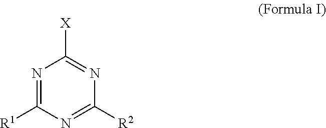

or a pharmaceutically acceptable salt thereof;
wherein R¹ is 4-aminoindole, 5-aminoindole, 6-aminoindole, or 7-aminoindole; R² is 4-aminoindole, 5-aminoindole, 6-aminoindole, or 7-aminoindole; and X is H, halo, NO₂, NH₂, OH, or O-alkyl. Halo can be Cl, F, I or Br. In an embodiment of Formula I, R¹ and R² are 4-aminoindole, 5-aminoindole, 6-aminoindole, or 7-aminoindole and X is H, halo, NO₂, NH₂, OH, or O-alkyl. Halo can be Cl, F, I or Br, such as Cl.

Further provided is compound of the formulae (II)-(IIc):

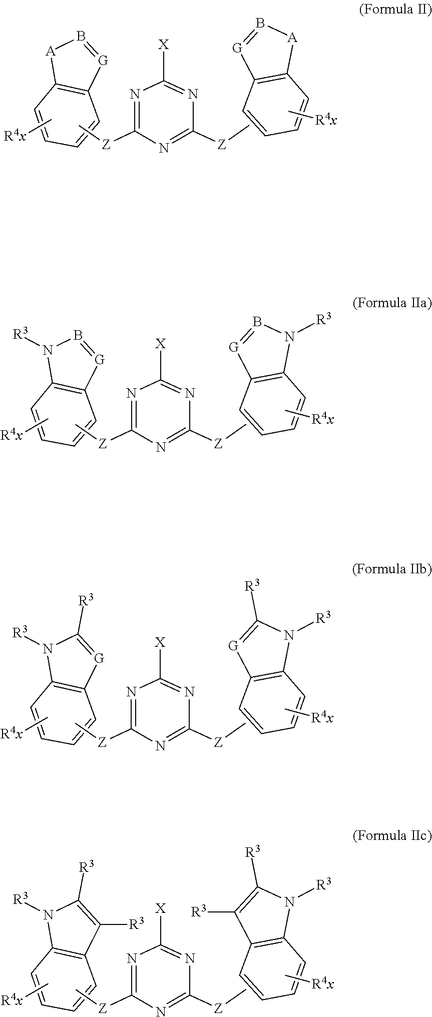

or a pharmaceutically acceptable salt thereof;
wherein:
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl;
each A is, independently, O, S or NR³, wherein R³ is H or alkyl;
each B is independently N or CR⁵, wherein R⁵ is H, acyl, amido, or alkyl; and each G is independently N or CR⁵, wherein R⁵ is H, acyl, amido, or alkyl.

Also provided is a pharmaceutical composition comprising the compound of Formula formulae (I) and (II)-(IIc) and a pharmaceutically acceptable carrier. Halo can be Cl, F, I or Br. In an embodiment of Formula I, R¹ and R² are 4-aminoindole and X is Cl.

Still further provided is a method of inhibiting tubulin-associated unit (tau) and/or alpha-synuclein (α-syn) protein aggregation in a subject having, or at risk for, tau and/or α-syn protein aggregation. The method comprises administering to the subject the above-described pharmaceutical composition in an amount effective to inhibit tau protein and/or α-syn protein aggregation. Tau can be tau isoform 2N4R and/or phosphorylated tau isoform 1N4R. The subject can have, or be at risk for, Alzheimer's disease or Parkinson's disease. The subject can have neuroblastoma, and the formation of α-syn inclusions can be inhibited. It also disaggregates amyloid plaques and paired helical filaments isolated from brains of humans with Alzheimer's disease (AD).

### FIGURES

FIG. 1 shows the chemical structures, physicochemical descriptors, and the effects of compounds on alpha-synuclein (α-syn) aggregation measured as fluorescence intensity using thioflavin T (ThT) assay.

FIG. 2 shows kinetics curves of the best compound exhibiting the lowest fluorescence intensity (i.e., compound 10). A di-substituted O-triazine, i.e., compound 7, was selected as a negative control. Compound 10 is a di-substituted N-triazine. Compounds 7 and 10 were tested at a final concentration of 100 M in the presence of α-syn at 6 μM. The curves represent the average of three technical replicates.

FIGS. 3A-3B shows the kinetic curves ((A) in hours; (B) in minutes) of tau isoform 2N4R fibril formation obtained with different compounds monitored using the thioflavin fluorescence assays. The compounds were tested at a final concentration of 100 M in the presence of tau 2N4R at 6 μM. CTRL, control; BG, background. The dotted lines represent mean±SEM from n=3 technical replicates.

FIGS. 4A-4B shows triazine derivative, compound 10, inhibits the α-syn and tau isoform 2N4R oligomerization in a dose-dependent manner. (A) α-syn (6 μM) and (B) Tau isoform 2N4R (6 μM) were cross-linked (PICUP assay) with di-substituted aminoindole triazine, compound 10, at the following concentrations: 50 μM (molar ratio 1:8), 12.5 μM (molar ratio~1:32), and 3.125 μM (molar ratio~1:128). Compound 7 was utilized as a negative control for both experiments.

FIGS. 5A-5F shows ultrastructural changes of α-syn, tau 2N4R, and p-tau 1N4R after incubation with Compound 10. Proteins (60 μM) were incubated with <0.25% DMSO or compound 10 at 600 μM (molar ratio 1:10) in 10 mM phosphate-buffered saline (PBS) (pH 7.4) for 24 hours at 37° C. prior to visualization by transmission electron microscopy (TEM). (A) α-Syn after incubation with dimethylsulfoxide (DMSO). (B) α-Syn after incubation with compound 10. (C) Tau (2N4R) after incubation with DMSO. (D) Tau (2N4R) after incubation with compound 10. (E) p-Tau (1N4R) after incubation with DMSO. (F) p-Tau (1N4R) after incubation with compound 10. Scale bare: 200 nm.

FIGS. 6A-6C shows compound 10 reduced inclusion formation in a dose-dependent manner using the M17D neuroblastoma cells that express inclusion-prone α-Synuclein (αS) 3K::yellow fluorescent protein (YFP). (A) M17D cells that express an αS3K::YFP fusion protein (dox-inducible) were treated with 0.1% DMSO (control) and compound 7 (negative control) at various concentrations at t=24 hrs. Cells were induced with dox at t=48 hours. Punctate YFP signals were measured and normalized to 0.1% DMSO at t=96 hours. The cell confluence percentage was plotted. For the first two graphs, four independent experiments (N=4; n=24; except DMSO, n=12) were performed. Total aS/calnexin ratios were plotted following Western blot (WB) analyses, normalized to 0.1% DMSO (N=4; n=24, except DMSO, n=4). Data are presented as fold-change relative to 0.1% DMSO. Ordinary one-way ANOVA plus Dunnett's multiple comparison test (*, p<0.1, **, p<0.01, ***, p<0.001, ****, p<0.0001). (B) Same as panel (A), but with compound 10. (C) shows control (DMSO) image, whereas (D) shows treated (20 M compound 10) image.

FIGS. 7A-7D shows ultra-structural changes of Aβ-plaques and paired helical filaments after incubation with DMSO (control) or compound 10. Extracted plaques from Alzheimer's brains were incubated with 1.5% DMSO (control) or compound 10 (50 μM) in 10 mM PBS buffer (pH 7.4) for 120 hours at 37° C. prior to visualization by transmission electron microscopy. (A) Aβ-plaque in the presence of DMSO. (B) Aβ-plaque in the presence of compound 10. (C) Paired helical filaments in the presence of DMSO. (D) Paired helical filaments in the presence of compound 10. Scale bare: 200 nm.

### DESCRIPTION

The present disclosure is predicated, at least in part, on the discovery of compounds that affect two prone-to-aggregate proteins—alpha-synuclein (α-syn) and the 2N4R isoform of tau (tau 2N4R). α-syn, when misfolded, results in Lewy body inclusions that limit motor movement in Parkinson's disease (PD). Compounds with a triazine linker were synthesized and evaluated for their effectiveness in reducing protein aggregation involving α-syn and tau 2N4R. One compound, in particular, is effective in reducing fibrillization and oligomerization of α-syn and tau 2N4R in a dose-dependent manner and can decrease the inclusion and confluence of α-syn M17D neuroblastoma cells in a concentration-dependent manner.

In view of the foregoing, provided is a compound of the formula:

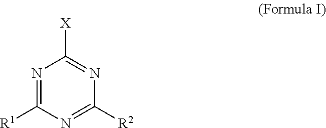

or a pharmaceutically acceptable salt thereof;
wherein R¹ is 4-aminoindole, 5-aminoindole, 6-aminoindole, or 7-aminoindole; R² is 4-aminoindole, 5-aminoindole, 6-aminoindole, or 7-aminoindole; and X is H, halo, NO₂, NH₂, OH, or O-alkyl. Halo can be Cl, F, I or Br. In an embodiment of Formula I, R¹ and R² are 4-aminoindole, 5-aminoindole, 6-aminoindole, or 7-aminoindole and X is H, halo, NO₂, NH₂, OH, or O-alkyl. Halo can be Cl, F, I or Br, such as Cl.

Also provided are compounds of the formula:

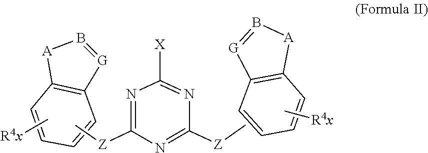

or a pharmaceutically acceptable salt thereof;
such as:

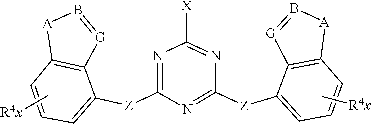

or a pharmaceutically acceptable salt thereof;
wherein:
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl;
each A is, independently, O, S or NR³, wherein R³ is H or alkyl;
each B is independently N or CR⁵, wherein R⁵ is H, acyl, amido, or alkyl; and
each G is independently N or CR⁵, wherein R⁵ is H, acyl, amido, or alkyl.

In the compounds of the formula (II), each Z can independently be O or NH. Alternatively or in addition, each A can independently be NR³. In one example, each R³ can independently be H. In another example, each R³ can independently be alkyl, such as C₁-C₆ alkyl or C₁-C₃ alkyl, such as methyl or ethyl. Alternatively, each A can independently be O. Alternatively, each A can independently be S. Alternatively or in addition, each B can independently be CR⁵, such as CH. Alternatively, each B can independently be N. Alternatively or in addition, each G can independently be CR⁵, such as CH. Alternatively each G can independently be N. Alternatively or in addition, each R⁴ can independently be H. Alternatively or in addition X can be H.

Also provided are compounds of the formula:

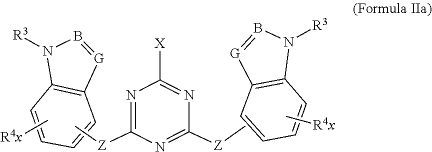

or a pharmaceutically acceptable salt thereof;
such as:

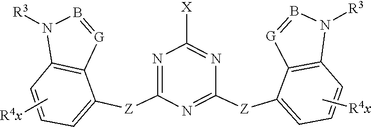

or a pharmaceutically acceptable salt thereof;
wherein:
each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl;
each B is independently N or CR⁵, wherein R⁵ is H, acyl, amido, or alkyl; and
each G is independently N or CR⁵, wherein R⁵ is H, acyl, amido, or alkyl.

In the compounds of the formula (IIa), each Z can independently be O or NH. Alternatively or in addition, each R³ can independently be H. In another example, each R³ can independently be alkyl, such as C₁-C₆ alkyl or C₁-C₃ alkyl, such as methyl or ethyl. Alternatively or in addition, each B can independently be CR⁵, such as CH. Alternatively, each B can independently be N. Alternatively or in addition, each G can independently be CR⁵, such as CH. Alternatively, each B can independently be N. Alternatively or in addition, each R⁴ can independently be H. Alternatively or in addition, X can be H.

Also provided are compounds of the formula:

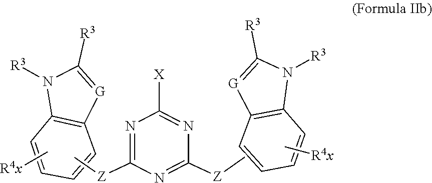

or a pharmaceutically acceptable salt thereof; such as:

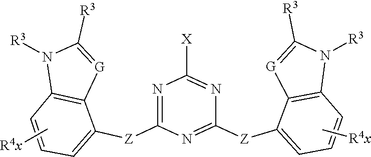

or a pharmaceutically acceptable salt thereof;
wherein:
wherein each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl; and
each G is independently N or CR⁵, wherein R⁵ is H, acyl, amido, or alkyl.

In the compounds of the formula (IIb), each Z can independently be O or NH. Alternatively or in addition, each R³ can independently be H. In another example, each R³ can independently be alkyl, such as C₁-C₆ alkyl or C₁-C₃ alkyl, such as methyl or ethyl. Alternatively or in addition, each G can independently be CR⁵, such as CH. Alternatively each G can independently be N. Alternatively or in addition, each R⁴ can independently be H. Alternatively or in addition, X can be H.

Also provided are compounds of the formula:

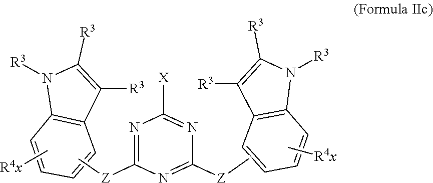

or a pharmaceutically acceptable salt thereof; such as:

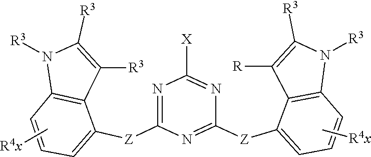

or a pharmaceutically acceptable salt thereof;
wherein:
each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl.

In the compounds of the formula (IIc), each Z can independently be O or NH. Alternatively or in addition, each R³ can independently be H. In another example, each R³ can independently be alkyl, such as C₁-C₆ alkyl or C₁-C₃ alkyl, such as methyl or ethyl. Alternatively or in addition, each R⁴ can independently be H. Alternatively or in addition, X can be H.

Thus, for example, in each of the compounds of the formula (II), (IIa), and (IIb), each group of the formula:

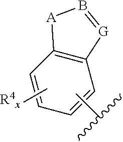

can independently be a group of the formula:

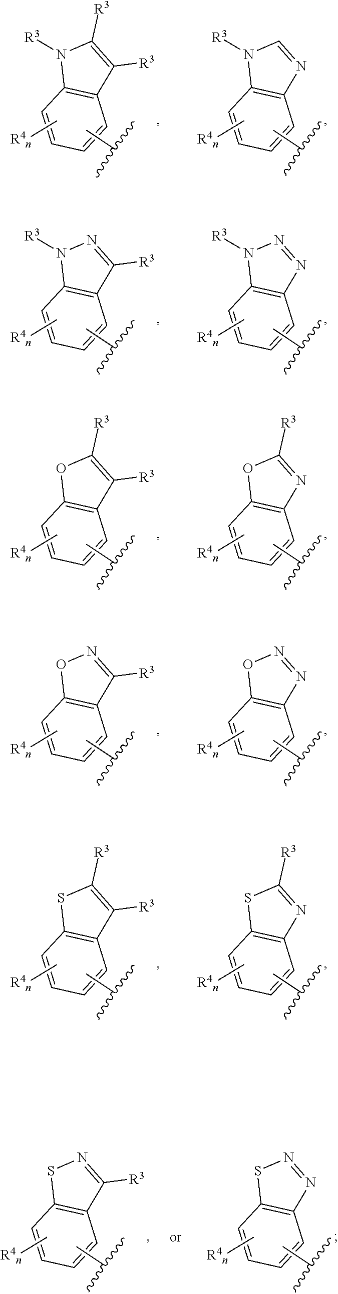

wherein:
each R³ is independently H or alkyl;
each x is independently 1, 2, or 3; and
each R⁴ is independently H, halo, nitro, cyano, alkoxy, amino or two R⁴ groups and the carbon atoms to which they are attached can form an aryl or a heterocyclyl group, such as:

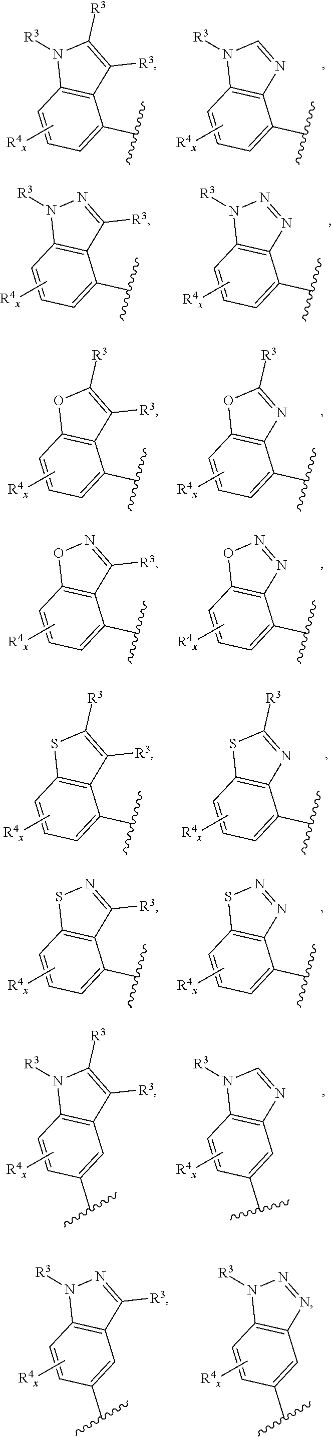

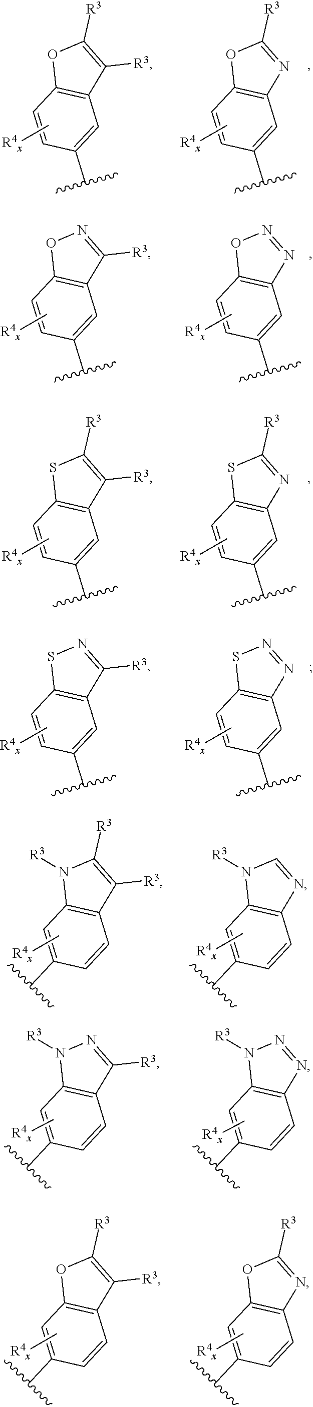

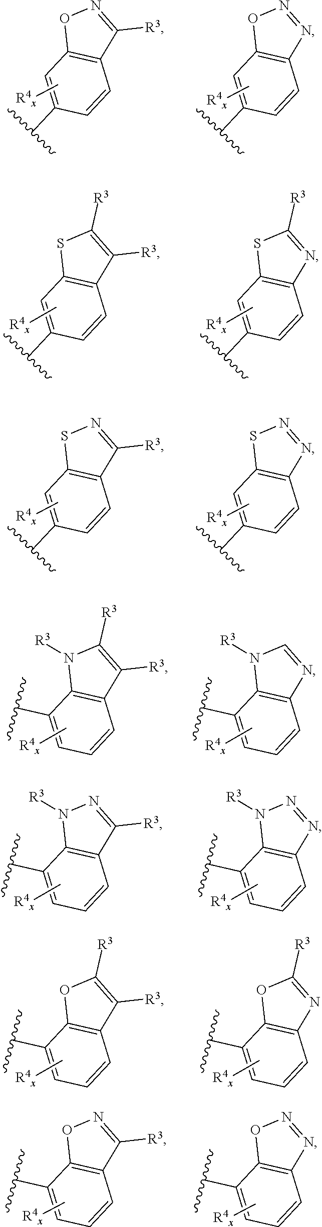

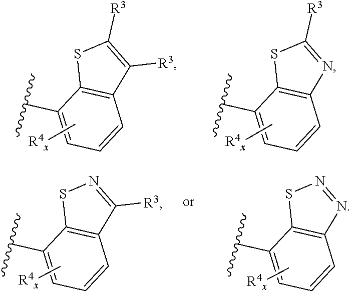

Each R³ can independently be H. In another example, each R³ can independently be alkyl, such as C₁-C₆ alkyl or C₁-C₃ alkyl, such as methyl or ethyl. Alternatively or in addition, each R⁴ can independently be H.

The above compounds include isotopic variants and compounds in which one or more hydrogen atoms have been substituted with deuterium. The compounds may contain one or more chiral centers or may otherwise be capable of existing as multiple stereoisomers. In embodiments, the compounds are not limited to any particular stereochemical requirement, and the compounds, and compositions, methods, uses, and medicaments that include them may be optically pure, or may be any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configurations at one or more other chiral centers.

Similarly, the compounds may include geometric centers, such as cis, trans isomers, diastereomers, enantiomers, and E and Z double bonds. In other embodiments, the compounds are not limited to any particular geometric isomer requirement, and the compounds, and compositions, methods, uses, and medicaments that include them may be pure, or may be any of a variety of geometric isomer mixtures. Such mixtures of geometric isomers may include a single configuration at one or more double bonds and chiral carbons, while including mixtures of geometry at one or more other double bonds and chiral carbons.

The terms “salts” and “pharmaceutically acceptable salts” refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference for its teachings regarding same.

The term “solvate” means a compound, or a salt thereof, that further includes a stoichiometric or non-stoichiometric amount of solvent bound by non-covalent intermolecular forces. Where the solvent is water, the solvate is a hydrate.

The above compounds, and pharmaceutically acceptable salts and solvates thereof, can be synthesized in accordance with methods known in the art and exemplified herein. See, e.g., Example 1.

The compounds can be used to inhibit the aggregation of proteins prone to aggregate in a state of disease. The protein prone to aggregate can be islet amyloid polypeptide, amyloid-β, α-synuclein, tubulin associated unit (tau), or transthyretin. The tau can be tau isoform 2N4R or 1N4R. The disease can be AA amyloidosis, Alzheimer's disease, monoclonal immunoglobulin light-chain amyloidosis, Huntington's disease, Parkinson's disease, Creutzfeldt-Jacob disease, prion disorders, amyotrophic lateral sclerosis, type 2 diabetes, or transthyretin amyloidosis.

The compounds can be used to inhibit tau protein aggregation in tauopathies. Tauopathies are a group of disorders that result from abnormal tau phosphorylation, abnormal levels of tau, abnormal tau splicing, and mutations in the tau gene, for example. Neurodegenerative diseases have been classified based on this protein accumulation. Tauopathies encompass more than 20 clinicopathological conditions, including Alzheimer's disease (AD), which is the most common tauopathy. Other tauopathies include, but are not limited to, familial AD, primary age-related tauopathy (PART), Creutzfeldt-Jacob disease, dementia pugilistica, Gerstmann-Straussler-Scheinker disease (GSS), inclusion-body myositis, cortico-basal degeneration (CBD), Picks disease (PiD), progressive supranuclear palsy (also known as Steele, Richardson, and Olszewski disorder), Down syndrome, Parkinsonism with dementia, myotonic dystrophy, prion protein cerebral amyloid angiopathy, traumatic brain injury (TBI), amyotrophic lateral sclerosis (ALS), Parkinsonism-dementia complex of Guam, non-Guamanian motor neuron disease with neurofibrillary tangles, argyrophilic grain disease, diffuse neurofibrillary tangles with calcification, frontotemporal dementia with Parkinsonism linked to chromosome 17 (FTDP-17), Haller-vorden-Spatz disease, multiple system atrophy (MSA), Niemann-Pick disease type C, pallido-ponto-nigral degeneration, progressive subcortical gliosis, progressive supranuclear palsy (PSP), subacute sclerosing panencephalitis, tangle predominant dementia, postencephalitic Parkinsonism, myotonic dystrophy, subacute sclerosis panencephalopathy, mutations in LRRK2, chronic traumatic encephalopathy (CTE), familial British dementia, familial Danish dementia, other frontotemporal lobar degenerations, Guadeloupean Parkinsonism, neurodegeneration with brain iron accumulation, SLC9A6-related mental retardation, white matter tauopathy with globular glial inclusions, epilepsy, Lewy body dementia (LBD), mild cognitive impairment (MCI), multiple sclerosis, Parkinson's disease, HIV-related dementia, adult onset diabetes, senile cardiac amyloidosis, glaucoma, ischemic stroke, psychosis in AD, Huntington's disease, and prion diseases with tangles. The majority of neurodegenerative diseases are characterized by the deposition of insoluble protein in cells of the neuromuscular system.

Further provided is a pharmaceutical composition comprising the compound of Formula I and a pharmaceutically acceptable carrier. Halo can be Cl, F, I or Br. In an embodiment of Formula I, R¹ and R² are 4-aminoindole, 5-aminoindole, 6-aminoindole, or 7-aminoindole and X is H, halo (e.g., Cl), NO₂, NH₂, OH, or O-alkyl. The compounds can be formulated as pharmaceutical compositions comprising a pharmaceutically acceptable carrier using methods well-known in the art. “Carrier” is used generically herein to refer to pharmaceutically acceptable carriers, diluents, adjuvants, and excipients. See, e.g., Remington: The Science and Practice of Pharmacy, 23^{rd} edition, Oct. 30, 2020, Adeboye Adejare, ed. Accordingly, further provided is a pharmaceutical composition comprising the compound of formula I and a pharmaceutically acceptable carrier.

Still further provided is a method of inhibiting tubulin-associated unit (tau) and/or alpha-synuclein (α-syn) protein aggregation in a subject having, or at risk for, tau and/or α-syn protein aggregation. The method comprises administering to the subject the above-described pharmaceutical composition in an amount effective to inhibit tau protein and/or α-syn protein aggregation. Tau can be tau isoform 2N4R and/or phosphorylated tau isoform 1N4R. The subject can have, or be at risk for, Alzheimer's disease or Parkinson's disease. The subject can have neuroblastoma, and the formation of α-syn inclusions can be inhibited.

Any suitable route of administration can be used in the above methods. Examples include, but are not limited to, oral, parenteral, intravenous, intracranial, intracerebroventricular, and intracerebral. An effective amount can be determined by one of ordinary skill in the art using dosage range determining methods known in the art. Typically, a physician (or veterinarian for non-human subjects) will determine the actual dosage, which will be most suitable for an individual subject. The specific dose level and frequency of dosage for an individual may be varied and will depend upon a variety of factors including the activity of the specific compound employed, the metabolic stability and length of action of that compound, the age, body weight, general health, gender, diet, mode and time of administration, rate of excretion, other administered drugs, and the severity of the particular condition. The compound/compositions described herein can be administered with other biologically active compounds as appropriate.

The terms “substituted,” “substituent,” and “functional group” refer to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The term “alkyl” as used herein refers to substituted or unsubstituted straight chain and branched mono- or divalent alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and ante-isoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched mono- or divalent alkenyl groups and cycloalkenyl groups having at least one double bond and having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkenyl groups include those with from 1 to 8 carbon atoms such as —CH═CH—, —CH═CHCH₃, and —CH₂CH═CHCH₂— groups, wherein the double bonds can have an E- or Z-configuration. And when there are multiple bonds, each double bond can, independently, have an E- or a Z-configuration. Examples of branched alkenyl groups include, but are not limited to, —CH═C(CH₃)— and CH₂C═CH(CH₃) groups. Representative substituted alkenyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups can have any number of carbon atoms, e.g., 3 to 8 carbon atoms (C₃-C₈), 3 to 6 carbon atoms (C₃-C₆), and 4 to 8 carbon atoms (C₄-C₅). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “cycloalkylalkyl” as used herein refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “alkylcycloalkyl” as used herein refers to substituted or unsubstituted cycloalkyl groups as defined herein in which a hydrogen of a cycloalkyl group as defined herein is replaced with a bond to an alkyl group as defined herein. Representative alkylcycloalkyl groups include, but are not limited to, alkylcyclopropyl.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. “Aryl” and the phrase “aryl group” includes fused ring species including those that include fused aromatic and non-aromatic groups. Accordingly, “aryl” and the phrase “aryl group” include groups of the formula:

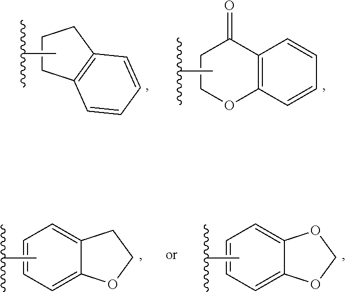

each of which can be substituted or unsubstituted, such as hydroxy substituted.

Representative substituted aryl groups can be mono-substituted or substituted more than once, such as, but not limited to, 2-, 3—, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” refers to substituted or unsubstituted aromatic and non-aromatic ring compounds containing 3 or more ring members, of which one or more (e.g., 1, 2 or 3) is a heteroatom such as, but not limited to, N, O, and S. Thus, a heterocyclyl can be a cycloheteroalkyl or a heteroaryl or, if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-ring with two carbon atoms and three heteroatoms, a 6-ring with two carbon atoms and the heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds, such as in the group 3,6-dihydro-2H-pyran and 3,4-dihydro-2H-pyran, having the formula:

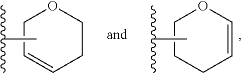

respectively, each of which can be substituted.

A heteroaryl ring is an embodiment of a heterocyclyl group. The phrase “heterocyclyl group” includes fused ring species including those that include fused aromatic and non-aromatic groups. Representative heterocyclyl groups include, but are not limited to tetrahydro-2H-thiopyran-1,1-dioxide, having the formula:

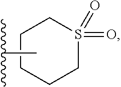

which can be substituted, 4a,5,6,7-tetrahydro-4H-pyrrolo[1,2-d][1,3,4]oxadiazinyl, having the formula:

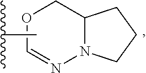

which can be substituted, pyrrolidinyl, pyrrolidinone (e.g., pyrrolidin-2-one), azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, imidazo[1,2-a]pyridinyl, having the formula:

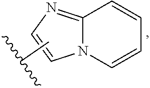

which can be substituted, triazyolyl, tetrazolyl, benzoxazolinyl, thiazolyl, benzthiazolinyl, and benzimidazolinyl groups. Examples of indolinonyl groups include groups having the general formula:

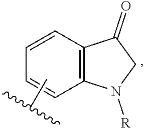

wherein R is as defined herein.

Examples of isoindolinonyl groups include groups having the general formula:

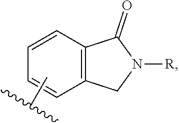

wherein R is as defined herein.

Examples of benzoxazolinyl groups include groups having the general formula:

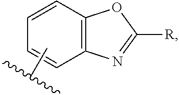

wherein R is as defined herein.

Examples of benzthiazolinyl groups include groups having the general formula:

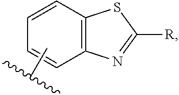

wherein R is as defined herein.

In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl group).

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” refers to alkyl groups as defined herein in which a hydrogen or carbon bond of an alkyl group as defined herein is replaced with a bond to a heterocyclyl group as defined herein and the alkyl group is attached to an oxygen. Representative heterocyclylalkoxy groups include, but are not limited to, —O—(CH₂)_{q}heterocyclyl, wherein q is an integer from 1 to 5. In some embodiments, heterocyclylalkoxy groups include —O—(CH₂)_{q}morpholinyl such as —O—CH₂CH₂-morpholine.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “amine,” “amine group,” “amino,” and “amino group” refer to a substituent of the form —NH₂, —NHR, —NR₂, or —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group.

An “alkylamino” group includes a monoalkylamino, dialkylamino, and trialkylamino group. An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The term “amido” refers to a group of the formula —C(O)NR₂, wherein R is defined herein.

The terms “halo,” “halogen,” and “halide” group, by themselves or as part of another substituent, mean, unless otherwise stated, a fluorine, chlorine, bromine, or iodine atom.

The term “haloalkyl” group includes mono-halo alkyl groups, poly-halo alkyl groups, wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The terms “treat,” “treating,” “treated,” and “treatment” (with respect to a disease or condition) are used to describe an approach for obtaining beneficial or desired results, preferably clinical results, and include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

An “effective amount” refers to any amount that is sufficient to achieve a desired biological effect. Combined with the teachings provided herein, by choosing among the various active conjugates or compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation. A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

A “therapeutically effective amount” (or “effective amount”) of a compound, with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

For any compound a therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface. Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. A pharmaceutical preparation for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, and sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., EDTA for neutralizing internal acid conditions, or can be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., J Appl Biochem 4:185-189 (1982). Other polymers that could be used are poly-1,3-dioxolane and poly-1,3,6-tioxocane. For pharmaceutical usage, as indicated above, polyethylene glycol moieties are suitable.

The location of release of a compound hereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

The compound can be included in the formulation as fine multi-particulates in the form of granules or pellets of particle size about 1 mm. The formulation of the material for capsule administration could also be as a powder, lightly compressed plugs or even as tablets. Therapeutic agent could be prepared by compression.

Colorants and flavoring agents may all be included. For example, the compound can be formulated (such as by liposome or microsphere encapsulation) and then further contained within an edible product, such as a refrigerated beverage containing colorants and flavoring agents.

One may dilute or increase the volume of the compound with an inert material. These diluents can include carbohydrates, especially mannitol, a-lactose, anhydrous lactose, cellulose, sucrose, modified dextrans and starch. Certain inorganic salts also can be used as fillers including calcium triphosphate, magnesium carbonate and sodium chloride. Some commercially available diluents are Fast-Flo, Emdex, STA-Rx 1500, Emcompress and Avicell.

Disintegrants can be included in the formulation of therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders, and these can include powdered gums such as agar, Karaya or tragacanth. Alginic acid and its sodium salt are also useful as disintegrants.

Binders can be used to hold the compound together to form a hard tablet and can include materials from natural products such as acacia, tragacanth, starch and gelatin. Others include methyl cellulose (MC), ethyl cellulose (EC) and carboxymethyl cellulose (CMC). PVP and hydroxypropylmethyl cellulose (HPMC) can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents, which can be used, include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well-defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator, can be formulated containing a powder mix of the compound and a suitable powder base, such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins,” Proceedings of Symposium on Respiratory Drug Delivery II, Keystone, Colorado, March, (recombinant hepatocyte growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same), issued Sep. 19, 1995, to Wong et al.

Contemplated for use are a wide range of mechanical devices designed for pulmonary delivery of therapeutic products including, but not limited to, nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering therapeutic product to the nose, without the necessity for deposition of the product in the lung. Formulations for nasal delivery include those with dextran or cyclodextran.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Alternatively, the active compounds can be in powder form for constitution with a suitable vehicle, e.g., sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosolized, pelleted for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-1533 (1990).

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Pharmaceutical compositions contain an effective amount of a compound as described herein and optionally one or more other therapeutic agents included in a pharmaceutically acceptable carrier. The term “pharmaceutically acceptable carrier” means one or more compatible solid or liquid fillers, diluents or encapsulating substances, which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction, which would substantially impair the desired pharmaceutical efficiency.

Therapeutic agent(s), including specifically, but not limited to, a compound, can be provided in particles. “Particles” means nanoparticles or microparticles (or in some instances larger particles) that can consist in whole or in part of the compound or the other therapeutic agent(s). The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:5823-2787 (1993), the teachings of which are specifically incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Therapeutic agent(s) can be contained in controlled-release systems. The term “controlled release” refers to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) refers to a drug formulation that provides for gradual release of a drug over an extended period of time, and that can result in substantially constant blood levels of a drug over an extended time period. The term “delayed release” refers to a drug formulation in which there is a time delay between administration of the formulation and the release of the drug therefrom. “Delayed release” may or may not involve gradual release of drug over an extended period of time, and thus may or may not be “sustained release.”

Use of a long-term, sustained-release implant can be particularly suitable for treatment of chronic conditions. “Long-term” release means that the implant is constructed and arranged to deliver therapeutic levels of the active ingredient for at least 7 days, and up to 30-60 days. Long-term sustained-release implants are well-known to those of ordinary skill in the art and include some of the release systems described above.

The term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound, which contains a basic or acidic moiety, by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, the disclosure of which is hereby incorporated by reference.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well-known to those skilled in the art. Parenteral administration of a compound is illustratively performed in the form of saline solutions or with the compound incorporated into liposomes. In cases where the compound, itself, is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

In the methods the individual components of a co-administration, or combination, can be administered by any suitable means, contemporaneously, simultaneously, sequentially in either order, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “patient” includes human and non-human animals such as companion animals (dogs and cats and the like) and livestock animals. Livestock animals are animals raised for food production. The patient to be treated is preferably a mammal, in particular a human.

The disclosure relates to, among other things, the following enumerated Embodiments, which listing does not represent an order of importance:

Embodiment 1. A compound of the formula:

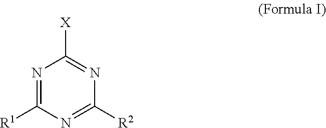

or a pharmaceutically acceptable salt thereof;
wherein R¹ is 4-aminoindole, 5-aminoindole, 6-aminoindole, or 7-aminoindole; R² is 4-aminoindole, 5-aminoindole, 6-aminoindole, or 7-aminoindole; and X is H, halo, NO₂, NH₂, OH, or O-alkyl.

Embodiment 2. The compound of Embodiment 1, wherein halo is Cl, F, I or Br.

Embodiment 3. The compound of Embodiment 1, wherein R¹ and R² are 4-aminoindole, 5-aminoindole, 6-aminoindole, or 7-aminoindole and X is H, halo, NO₂, NH₂, OH, or O-alkyl.

Embodiment 4. The compound of Embodiment 3, wherein X is halo and halo is Cl.

Embodiment 5. A compound of the formula:

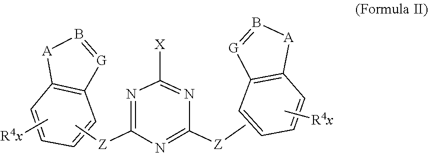

or a pharmaceutically acceptable salt thereof;
wherein:
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl;
each A is, independently, O, S or NR³, wherein R³ is H or alkyl;
each B is independently N or CR⁵, wherein R⁵ is H, acyl, amido, or alkyl; and each G is independently N or CR⁵, wherein R⁵ is H, acyl, amido, or alkyl.

Embodiment 6 relates to the compound of Embodiment 5, wherein the compound is of the formula:

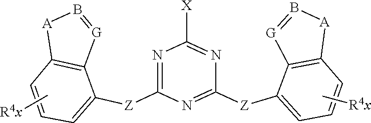

or a pharmaceutically acceptable salt thereof.

Embodiment 7. The compound of Embodiment 5 or 6, wherein each Z is independently O or NH.

Embodiment 8. The compound of Embodiment 5 or 6, wherein each A is independently NR³.

Embodiment 9. The compound of Embodiment 7, wherein each A is independently NR³.

Embodiment 10. The compound of Embodiment 8, wherein each R³ is H.

Embodiment 11. The compound of Embodiment 9, wherein each R³ is H.

Embodiment 12. The compound of Embodiment 8, wherein each R³ is independently C₁-C₆ alkyl.

Embodiment 13. The compound of Embodiment 9, wherein each R³ is independently C₁-C₆ alkyl.

Embodiment 14. The compound of Embodiment 5 or 6, wherein each A is O.

Embodiment 15. The compound of Embodiment 7, wherein each A is O.

Embodiment 16. The compound of Embodiment 5 or 6, wherein each A is S.

Embodiment 17. The compound of Embodiment 7, wherein each A is S.

Embodiment 18. The compound of 5 or 6, wherein each B is independently CR⁵.

Embodiment 19. The compound of Embodiment 7, wherein each B is independently CR⁵.

Embodiment 20. The compound of Embodiment 18, wherein CR⁵ is CH.

Embodiment 21. The compound of Embodiment 19, wherein CR⁵ is CH.

Embodiment 22. The compound of Embodiment 5 or 6, wherein each B is N.

Embodiment 23. The compound of Embodiment 7, wherein each B is N.

Embodiment 24. The compound of Embodiment 5 or 6, wherein each G is independently CR⁵.

Embodiment 25. The compound of Embodiment 7, wherein each G is independently CR⁵.

Embodiment 26. The compound of Embodiment 24, wherein CR⁵ is CH.

Embodiment 27. The compound of Embodiment 25, wherein CR⁵ is CH.

Embodiment 28. The compound of Embodiment 5 or 6, wherein each G is N.

Embodiment 29. The compound of Embodiment 7, wherein each G is N.

Embodiment 30. The compound of Embodiment 5 or 6, wherein each R⁴ is H.

Embodiment 31. The compound of Embodiment 7, wherein each R⁴ is H.

Embodiment 32. The compound of Embodiment 5 or 6, wherein X is H.

Embodiment 33. The compound of Embodiment 7, wherein X is H.

Embodiment 34. The compound of Embodiment 5, wherein the compound is of the formula:

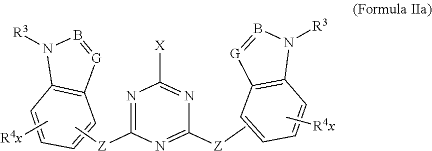

or a pharmaceutically acceptable salt thereof;
wherein:
each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl;
each B is independently N or CR⁵, wherein R⁵ is H, acyl, amido, or alkyl; and
each G is independently N or CR⁵, wherein R⁵ is H, acyl, amido, or alkyl.

Embodiment 35. The compound of Embodiment 34, wherein the compound is of the formula:

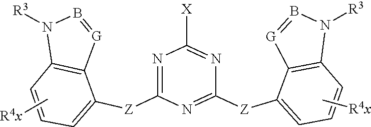

or a pharmaceutically acceptable salt thereof.

Embodiment 36. The compound of Embodiment 34 or 35, wherein each Z is independently O or NH.

Embodiment 37. The compound of Embodiment 34 or 35, wherein each R³ is H.

Embodiment 38. The compound of Embodiment 36, wherein each R³ is H.

Embodiment 39. The compound of Embodiment 34 or 35, wherein each R³ is independently C₁-C₆ alkyl.

Embodiment 40. The compound of Embodiment 36, wherein each R³ is independently C₁-C₆ alkyl.

Embodiment 41. The compound of Embodiment 34 or 35, wherein each B is independently CR⁵.

Embodiment 42. The compound of Embodiment 36, wherein each B is independently CR⁵.

Embodiment 43. The compound of Embodiment 34 or 35, wherein CR⁵ is CH.

Embodiment 44. The compound of Embodiment 36, wherein CR⁵ is CH.

Embodiment 45. The compound of Embodiment 34 or 35, wherein each B is N.

Embodiment 46. The compound of Embodiment 36, wherein each B is N.

Embodiment 47. The compound of Embodiment 34 or 35, wherein each G is independently CR⁵.

Embodiment 48. The compound of Embodiment 36, wherein each G is independently CR⁵.

Embodiment 49. The compound of Embodiment 47, wherein CR⁵ is CH.

Embodiment 50. The compound of Embodiment 48, wherein CR⁵ is CH.

Embodiment 51. The compound of Embodiment 34 or 35, wherein each G is N.

Embodiment 52. The compound of Embodiment 36, wherein each G is N.

Embodiment 53. The compound of 34 or 35, wherein each R⁴ is H.

Embodiment 54. The compound of Embodiment 36, wherein each R⁴ is H.

Embodiment 55. The compound of Embodiment 34 or 35, wherein X is H.

Embodiment 56. The compound of Embodiment 36, wherein X is H.

Embodiment 57. The compound of Embodiment 5, wherein the compound is of the formula:

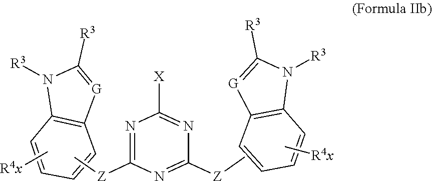

or a pharmaceutically acceptable salt thereof;
wherein:
wherein each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl; and
each G is independently N or CR⁵, wherein R⁴ is H, acyl, amido, or alkyl.

Embodiment 58. The compound of Embodiment 57, wherein the compound is of the formula:

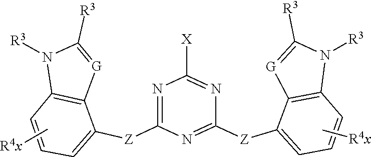

or a pharmaceutically acceptable salt thereof.

Embodiment 59. The compound of Embodiment 57 or 58, wherein each Z is independently O or NH.

Embodiment 60. The compound of Embodiment 57 or 58, wherein each R³ is H.

Embodiment 6l. The compound of Embodiment 59, wherein each R³ is H.

Embodiment 62. The compound of Embodiment 57 or 58, wherein each R³ is independently C₁-C₆ alkyl.

Embodiment 63. The compound of Embodiment 59, wherein each R³ is independently C₁-C₆ alkyl.

Embodiment 64. The compound of Embodiment 57 or 58, wherein each G is independently CR⁵.

Embodiment 65. The compound of Embodiment 59, wherein each G is independently CR⁵.

Embodiment 66. The compound of Embodiment 63, wherein CR⁵ is CH.

Embodiment 67. The compound of Embodiment 65, wherein CR⁵ is CH.

Embodiment 68. The compound of 57 or 58, wherein each G is N.

Embodiment 69. The compound of Embodiment 59, wherein each G is N.

Embodiment 70. The compound of Embodiment 57 or 58, wherein each R⁴ is H.

Embodiment 71. The compound of Embodiment 59, wherein each R⁴ is H.

Embodiment 72. The compound of Embodiment 57 or 58, wherein X is H.

Embodiment 73. The compound of Embodiment 59, wherein X is H.

Embodiment 74. The compound of Embodiment 5, wherein the compound is of the formula:

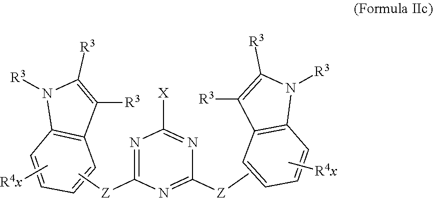

or a pharmaceutically acceptable salt thereof;
wherein:
each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl; and
each Z is independently 0 or NR³, wherein R³ is H or alkyl.

Embodiment 75. The compound of Embodiment 74, wherein the compound has the formula:

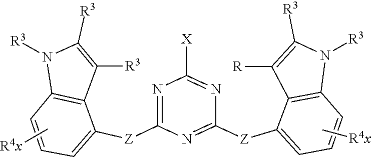

or a pharmaceutically acceptable salt thereof.

Embodiment 76. The compound of Embodiment 74 or 75, wherein each Z is independently O or NH.

Embodiment 77. The compound of Embodiment 74 or 75, wherein each R³ is H.

Embodiment 78. The compound of Embodiment 76, wherein each R³ is H.

Embodiment 79. The compound of Embodiment 74 or 75, wherein each R³ is independently C₁-C₆ alkyl.

Embodiment 82. The compound of Embodiment 76, wherein each R³ is independently C₁-C₆ alkyl.

Embodiment 81. The compound of Embodiment 74 or 75, wherein each R⁴ is H.

Embodiment 82. The compound of Embodiment 76, wherein each R⁴ is H.

Embodiment 83. The compound of Embodiment 74 or 75, wherein X is H.

Embodiment 84. The compound of Embodiment 76, wherein X is H.

Embodiment 85. The compound of Embodiment 5, wherein each group of the formula:

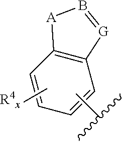

is independently a group of the formula:

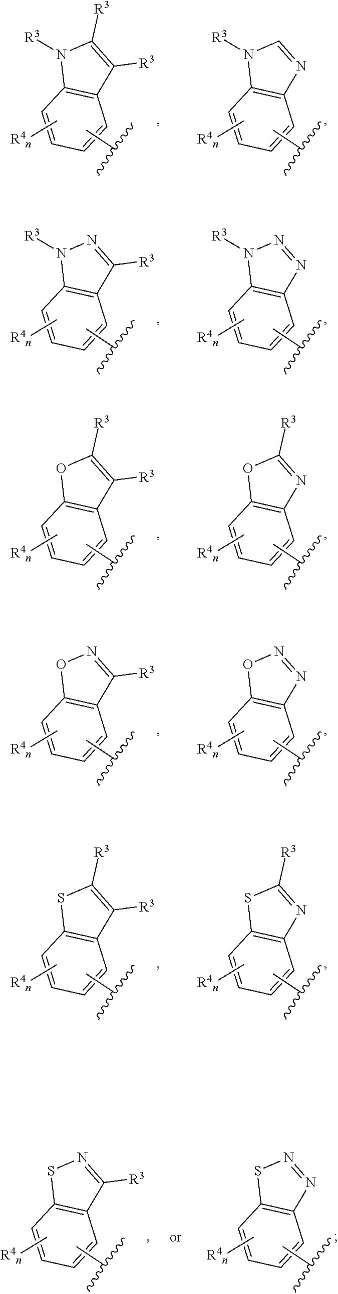

wherein:
each R³ is independently H or alkyl;
each x is independently 1, 2, or 3; and
each R⁴ is independently H, halo, nitro, cyano, alkoxy, amino or two R⁴ groups and the carbon atoms to which they are attached can form an aryl or a heterocyclyl group.

Embodiment 86. The compound of Embodiment 5, wherein each group of the formula:

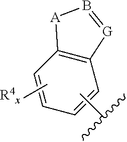

is independently a group of the formula:

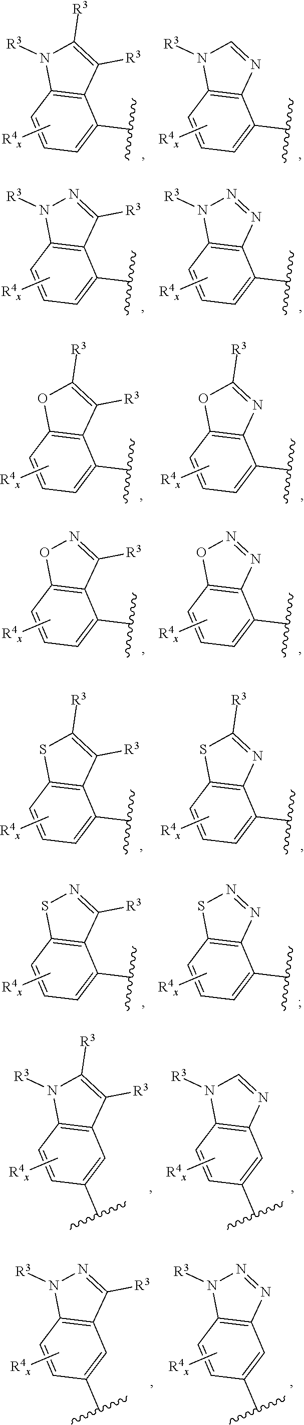

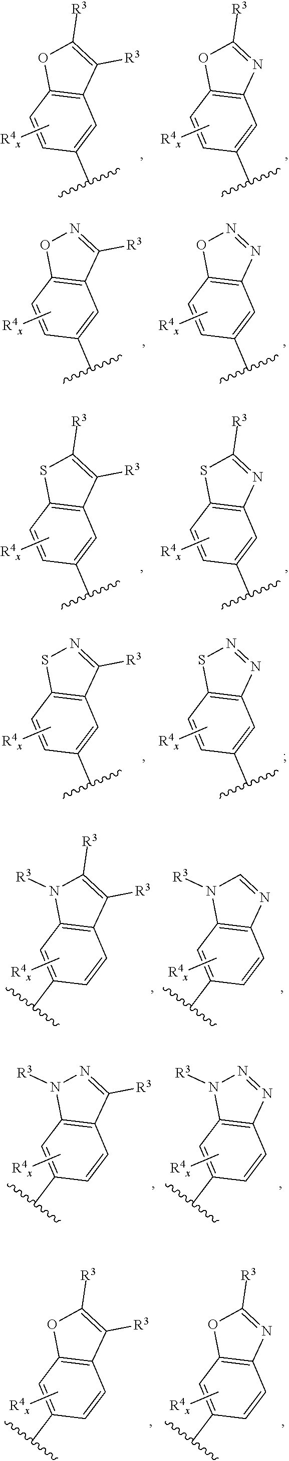

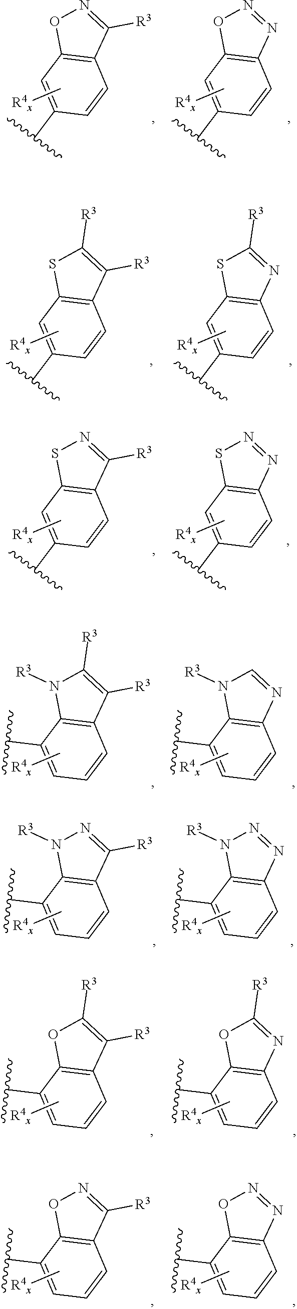

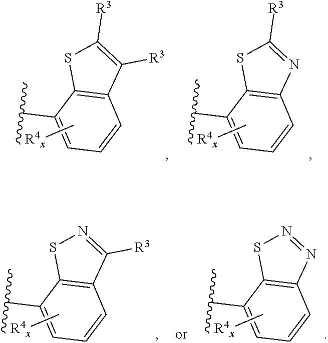

Embodiment 87. The compound of Embodiment 85 or 86, wherein each R³ is H.

Embodiment 88. The compound of Embodiment 85 or 86, wherein each R³ is independently C₁-C₆ alkyl.

Embodiment 89. The compound of Embodiment 85 or 86, wherein each R⁴ is independently H.

Embodiment 90. The compound of Embodiment 87, wherein each R⁴ is H.

Embodiment 91. The compound of Embodiment 88, wherein each R⁴ is H.

Embodiment 92. A pharmaceutical composition comprising the compound of any one of Embodiments 1-91 and a pharmaceutically acceptable carrier.

Embodiment 93. A method of inhibiting tubulin-associated unit (tau) and/or alpha-synuclein (α-syn) protein aggregation in a subject having, or at risk for, tau and/or α-syn protein aggregation, which method comprises administering to the subject the composition of Embodiment 92 in an amount effective to inhibit tau protein and/or α-syn protein aggregation, whereupon tau and/or α-syn protein aggregation is inhibited in the subject having, or at risk for tau and/or α-syn protein aggregation.

Embodiment 94. The method of Embodiment 93, wherein tau is tau isoform 2N4R.

Embodiment 95. The method of Embodiment 93, wherein tau is phosphorylated tau isoform 1N4R.

Embodiment 96. The method of Embodiment 93, wherein the subject has, or is at risk for, Alzheimer's disease.

Embodiment 97. The method of Embodiment 93, wherein the subject has, or is at risk for, Parkinson's disease.

Embodiment 98. The method of Embodiment 93, wherein the subject has neuroblastoma and the formation of α-syn inclusions is inhibited.

### EXAMPLES

The following examples serve to illustrate the present disclosure. The examples are not intended to limit the scope of the claimed invention in any way.

### Example 1: Synthesis

Chemical synthesis. All chemicals were obtained from conventional commercial sources and used without further purification unless otherwise noted. The ¹H NMR and ¹³C NMR chemical shifts are reported in parts per million relatives to the residual solvent protons.

Chemical and peptide source. DMSO and thioflavin-T (ThT) were purchased from Alfa Aesar (Ward Hill, MA). Procuration of recombinant α-syn was from rPeptide (WatKinsville, GA). The recombinant p-tau isoform 1N4R was prepared as published previously.^{31, 36} Concerning the preparation of tau 2N4R, bacterial expression plasmid consisting of the vector pRK172 carrying a cDNA encoding the human Tau 2N4R isoform was a kind gift of Dr. David Eliezer (Weill Cornell Medicine). For protein expression, E. coli BL21(DE3) cells were transformed with the plasmid and grown in LB media supplemented with ampicillin (100 μg/mL). Protein over-expression was induced by the addition of 1 mM IPTG for 4 h at 37° C., and cells were pelleted by centrifugation at 6,000 g for 15 min at 4° C. The cells were resuspended in lysis buffer (20 mM MES, 400 mM NaCl, 0.2 mM MgCl₂, 1 mM EGTA, protease inhibitor cocktail (P8340, Sigma Aldrich), 0.25 mg/mL lysozyme, and 1 μg/mL DNase I, pH 6.8) and lysed by a French press cell disruptor at 4° C., and the lysate was boiled for 20 min. Denatured proteins were pelleted by centrifugation at 30,000 g for 30 min at 4° C., and the supernatant was dialyzed overnight against cation exchange buffer (20 mM MES, 50 mM NaCl, 1 mM MgCl₂, 1 mM EGTA, 2 mM DTT, 0.1 mM PMSF, pH 6.8). The dialysate was loaded onto a HiPrep SP HP column, and proteins were eluted with a linear gradient ranging from 50 mM to 1 M NaCl. Fractions containing tau isoform 2N4R were pooled, and the resulting protein solution was dialyzed against PBS (pH 7.4) and stored at −80° C.

A novel series of 4, 5 and 6-mono/di/tri 0 and N substituted indolotriazines were synthesized and evaluated for their potential as anti-aggregation agents against alpha-synuclein (α-syn) and tubulin-associated unit (tau) protein isoform 2N4R. 2,4-dichloro-1,3,5-triazine and 2,4,6-trichloro-1,3,5-triazine easily undergo nucleophilic substitution (0 and N) reactions in presence of a base. To synthesize compounds 1-6 and 13-18, one equivalent of 4-, 5- and 6-amino/hydroxy indole were reacted with 2,4-dichloro-1,3,5-triazine and 2,4,6-trichloro-1,3,5-triazine respectively, in presence of a base: DIPEA (for hydroxyindoles) and NaHCO₃ (for aminoindoles) at 0° C. for 12 hours (Schemes 1 and 3). To synthesize the symmetrical di-substituted triazine derivatives 7-12 and 19-24, two equivalents of 4-, 5- and 6-amino/hydroxy indole were reacted with 2,4-dichloro-1,3,5-triazine and 2,4,6-trichloro-1,3,5-triazine, respectively, using the similar above-mentioned conditions, except the reaction was carried out at room temperature since substituted triazines have a higher 7-orbital electron density, therefore requiring elevated temperatures for nucleophilic substitution (Schemes 2 and 4). Following the same protocol, the trisubstituted indolotriazine derivative 25 was prepared by treating 3 equivalents of 4-aminoindole with 2,4,6-trichloro-1,3,5-triazine in presence of K₂CO₃ in THE under reflux conditions for 18 hours.

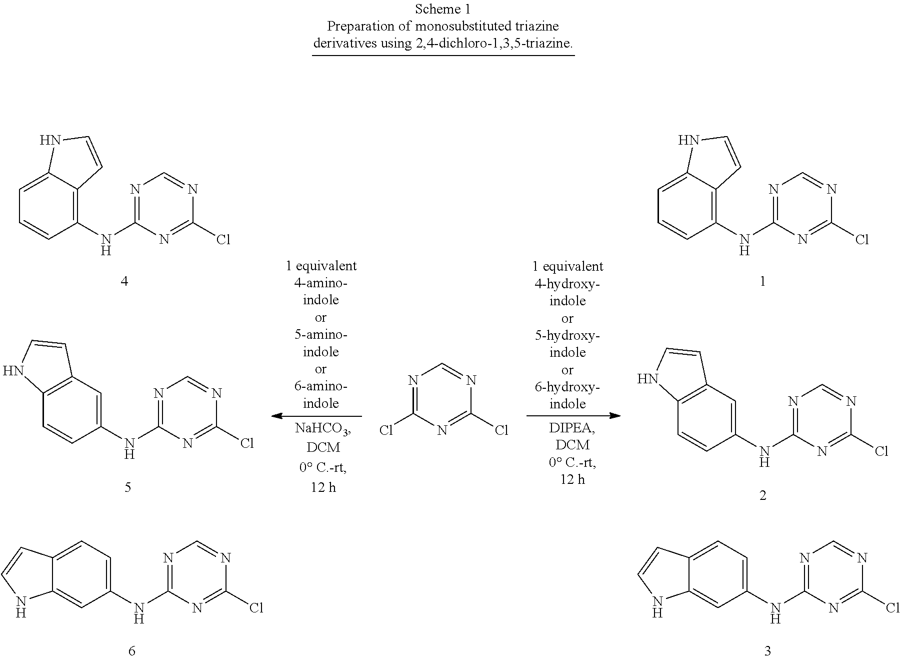

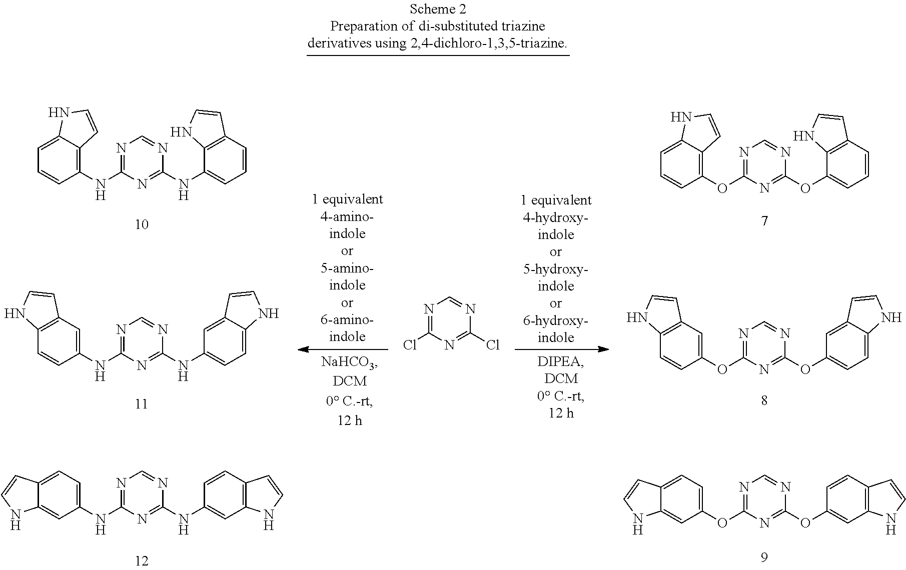

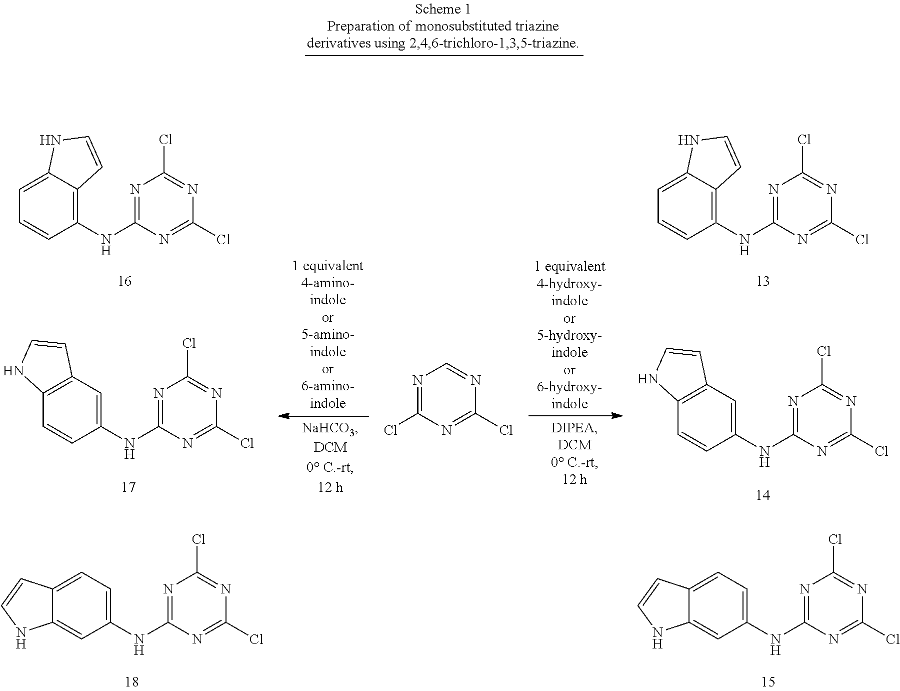

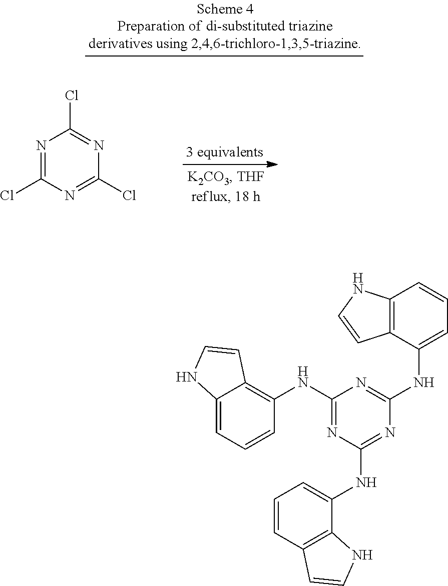 [SMILES: Clc1nc(Cl)nc(Cl)n1 ; c1cc(Nc2nc(Nc3cccc4[nH]ccc34)nc(Nc3cccc4[nH]ccc34)n2)c2[nH]ccc2c1]

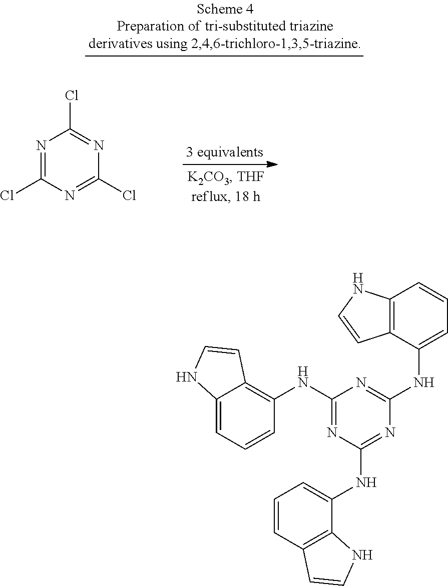 [SMILES: Clc1nc(Cl)nc(Cl)n1 ; c1cc(Nc2nc(Nc3cccc4[nH]ccc34)nc(Nc3cccc4[nH]ccc34)n2)c2[nH]ccc2c1]

4-[(4-chloro-1,3,5-triazin-2-yl)oxy]-1H-indole (Compound 1). ¹H NMR (500 MHz, DMSO) δ 11.43 (s, 1H), 8.95 (s, 1H), 7.41-7.34 (m, 2H), 7.12 (t, J=7.9 Hz, 1H), 6.89 (dd, J=7.7, 0.8 Hz, 1H). ¹³C NMR (126 MHz, DMSO) δ 171.9, 171.0, 170.6, 144.2, 138.6, 126.6, 121.7, 120.8, 110.9, 110.6, 98.3. HRMS (ESI) calcd for C₁₁H₇ClN₄O [M] 246.0308, found [M] 246.1281 and [M-1]⁺ 245.0938.

5-[(4-chloro-1,3,5-triazin-2-yl)oxy]-1H-indole (Compound 2). ¹H NMR (500 MHz, DMSO) δ 11.27 (s, 1H), 8.94 (s, 1H), 7.50-7.34 (m, 3H), 6.97 (dd, J=8.7, 2.3 Hz, 1H), 6.45 (t, J=1.0 Hz, 1H). ¹³C NMR (126 MHz, DMSO) δ 171.7 (2C), 170.5, 145.0, 134.4, 128.3, 127.6, 115.1, 112.6, 112.1, 101.9. HRMS (ESI) calcd for C₁₁H₇ClN₄O [M] 246.0308, found [M] 246.1285 and [M-1]⁺ 245.0944.

6-[(4-chloro-1,3,5-triazin-2-yl)oxy]-1H-indole (Compound 3). ¹H NMR (500 MHz, DMSO) δ 11.25 (s, 1H), 8.94 (s, 1H), 7.58 (d, J=8.5 Hz, 1H), 7.39 (dd, J=3.2, 2.4 Hz, 1H), 7.30 (d, J=2.1 Hz, 1H), 6.89 (dd, J=8.5, 2.2 Hz, 1H), 6.47 (s, 1H). ¹³C NMR (126 MHz, DMSO) δ 171.7, 171.5, 170.5, 146.8, 136.1, 127.0, 126.5, 121.1, 113.3, 104.5, 101.7. HRMS (ESI) calcd for C₁₁H₇ClN₄O [M] 246.0308, found [M+H]⁺ 247.0384.

N-(4-chloro-1,3,5-triazin-2-yl)-1H-indol-4-amine (Compound 4). 1H NMR (500 MHz, DMSO) δ 11.18 (s, 1H), 10.58 (s, 1H), 8.54 (d, J=35.5 Hz, 1H), 7.36-7.18 (m, 3H), 7.08 (d, J=7.8 Hz, 1H), 6.54 (s, 1H). ¹³C NMR (126 MHz, DMSO) δ 168.2, 167.7, 164.9, 137.3, 129.1, 125.4, 123.0, 121.3, 114.2, 109.7, 100.1. HRMS (ESI) calcd for C₁₁H₈ClN₅ [M]245.0468, found [M+H]⁺ 246.0543.

N-(4-chloro-1,3,5-triazin-2-yl)-1H-indol-5-amine (Compound 5). ¹H NMR (500 MHz, DMSO) δ 11.10 (d, J=11.8 Hz, 1H), 10.55 (d, J=7.2 Hz, 1H), 8.53 (d, J=10.3 Hz, 1H), 7.77 (dd, J=39.6, 2.0 Hz, 1H), 7.41-7.31 (m, 2H), 7.23 (td, J=8.8, 2.1 Hz, 1H), 6.41 (dt, J=4.9, 2.3 Hz, 1H). ¹³C NMR (126 MHz, DMSO) δ 170.1, 169.3, 168.3, 167.4, 164.2, 164.2, 133.9, 129.6, 129.3, 127.9, 126.8, 117.4, 117.1, 114.1, 113.7, 111.8, 111.7, 101.7. HRMS (ESI) calcd for C₁₁H₈ClN₅ [M] 245.0468, found [M+H]⁺ 246.0546.

N-(4-chloro-1,3,5-triazin-2-yl)-1H-indol-6-amine (Compound 6). ¹H NMR (500 MHz, DMSO) δ 11.13 (d, J=27.7 Hz, 1H), 10.66 (d, J=7.0 Hz, 1H), 8.57 (d, J=22.8 Hz, 1H), 7.83 (d, J=36.0 Hz, 1H), 7.48 (t, J=7.9 Hz, 1H), 7.30 (s, 1H), 7.16 (ddd, J=12.3, 8.3, 1.9 Hz, 1H), 6.38 (s, 1H). ¹³C NMR (126 MHz, DMSO) δ 170.1, 169.3, 168.3, 167.4, 163.9, 136.2, 131.9, 131.6, 126.2, 125.4, 125.2, 120.4, 120.3, 114.6, 114.5, 105.0, 104.8, 101.5. HRMS (ESI) calcd for C₁₁H₈ClN₅ [M] 245.0468, found [M+H]⁺ 246.0550.

4-{[4-(1H-indol-4-yloxy)-1,3,5-triazin-2-yl]oxy}-1H-indole(Compound 7). ¹H NMR (500 MHz, DMSO) δ 11.36 (s, 2H), 8.70 (s, 1H), 7.42-7.27 (m, 4H), 7.10 (t, J=7.9 Hz, 2H), 6.86 (dd, J=7.7, 0.8 Hz, 2H), 6.24 (s, 2H). ¹³C NMR (126 MHz, DMSO) δ 172.5, 170.9, 144.5, 138.5, 126.4, 121.7, 121.1, 111.1, 110.2, 98.2. HRMS (ESI) calcd for C₉H₃N₅O₂[M] 343.1069, found [M+H]⁺ 344.1153.

5-{[4-(1H-indol-5-yloxy)-1,3,5-triazin-2-yl]oxy}-1H-indole (Compound 8). ¹H NMR (500 MHz, DMSO) δ 11.21 (s, 2H), 8.70 (s, 1H), 7.60-7.24 (m, 6H), 6.94 (d, J=8.7 Hz, 2H), 6.42 (s, 2H). ¹³C NMR (126 MHz, DMSO) δ 173.0, 170.7, 145.2, 134.2, 128.2, 127.4, 115.5, 112.4, 112.2, 101.9. HRMS (ESI) calcd for C₁₉H₁₃N₅O₂ [M] 343.1069, found [M+H]⁺ 344.1142.

6-{[4-(1H-indol-6-yloxy)-1,3,5-triazin-2-yl]oxy}-1H-indole (Compound 9). ¹H NMR (500 MHz, DMSO) δ 11.19 (s, 2H), 8.72 (s, 1H), 7.55 (d, J=8.5 Hz, 2H), 7.37 (dd, J=3.1, 2.4 Hz, 2H), 7.26 (dt, J=2.1, 0.7 Hz, 2H), 6.87 (dd, J=8.5, 2.2 Hz, 2H), 6.45 (s, 2H). ¹³C NMR (126 MHz, DMSO) δ 172.9, 170.7, 147.1, 136.1, 126.8, 126.2, 120.9, 113.6, 104.6, 101.6. HRMS (ESI) calcd for C₁₉H₁₃N₅O₂ [M] 343.1069, found [M+H]⁺ 344.1147.

N2,N4-bis(1H-indol-4-yl)-1,3,5-triazine-2,4-diamine (Compound 10). ¹H NMR (500 MHz, DMSO) δ 11.18 (s, 2H), 10.58 (s, 2H), 8.77-8.27 (m, 2H), 7.42-7.16 (m, 6H), 7.07 (t, J=7.9 Hz, 2H), 6.54 (s, 2H). ¹³C NMR (126 MHz, DMSO) δ 168.23, 164.92, 137.34, 129.08, 125.43, 122.98, 121.29, 114.21, 109.76, 100.08. HRMS (ESI) calcd for C₁₉H₁₅N₇[M] 341.1389, found [M+H]⁺ 342.1469.

N2,N4-bis(1H-indol-5-yl)-1,3,5-triazine-2,4-diamine (Compound 11). ¹H NMR (500 MHz, DMSO) δ 11.10 (d, 2H), 10.55 (d, 2H), 8.52 (d, 2H), 7.76 (dd, 2H), 7.35 (m, 3H), 7.23 (m, 2H), 6.41 (dt, 2H). ¹³C NMR (126 MHz, DMSO) δ 168.3, 167.4, 129.6, 127.9, 126.8, 117.4, 117.1, 114.0, 113.7, 111.8, 101.7. HRMS (ESI) calcd for C₁₉H₁₅N₇[M] 341.1389, not detected.

N2,N4-bis(1H-indol-6-yl)-1,3,5-triazine-2,4-diamine (Compound 12). ¹H NMR (500 MHz, DMSO) δ 11.13 (d, J=27.8 Hz, 2H), 10.66 (d, J=7.4 Hz, 2H), 8.57 (d, J=23.0 Hz, 1H), 7.95-7.73 (m, 2H), 7.48 (t, J=7.9 Hz, 2H), 7.30 (q, J=2.6 Hz, 2H), 7.16 (ddd, J=12.3, 8.4, 1.9 Hz, 2H), 6.38 (t, J=2.0 Hz, 2H). ¹³C NMR (126 MHz, DMSO) δ 170.0, 169.3, 168.3, 167.4, 163.9, 136.2, 131.9, 131.6, 126.2, 120.3, 114.6, 114.5, 105.0, 104.8, 101.5. HRMS (ESI) calcd for C₁₉H₁₅N₇[M] 341.1389, not detected.

### Example 2 Effect of Different Triazine Derivatives on the Kinetics of α-Synuclein (α-Syn) Fibril Formation Monitored by Thioflavin-T (ThT) Fluorescence Assay

Structure-activity relationship (SAR): Concerning the impact of different substitutions on the triazine ring on kinetics of fibril formation was assessed using thioflavin T (ThT) assay by measuring the percentage fluorescence intensities (FI).

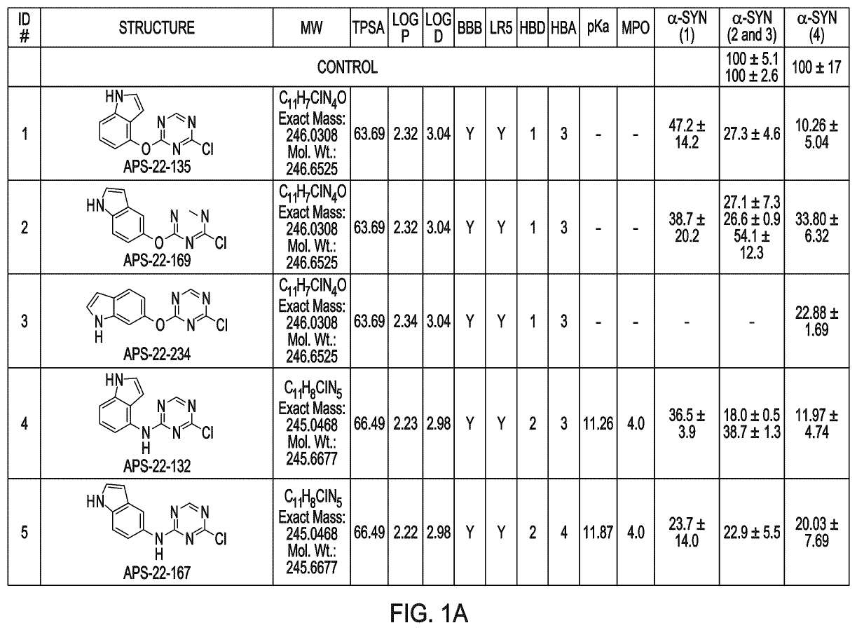

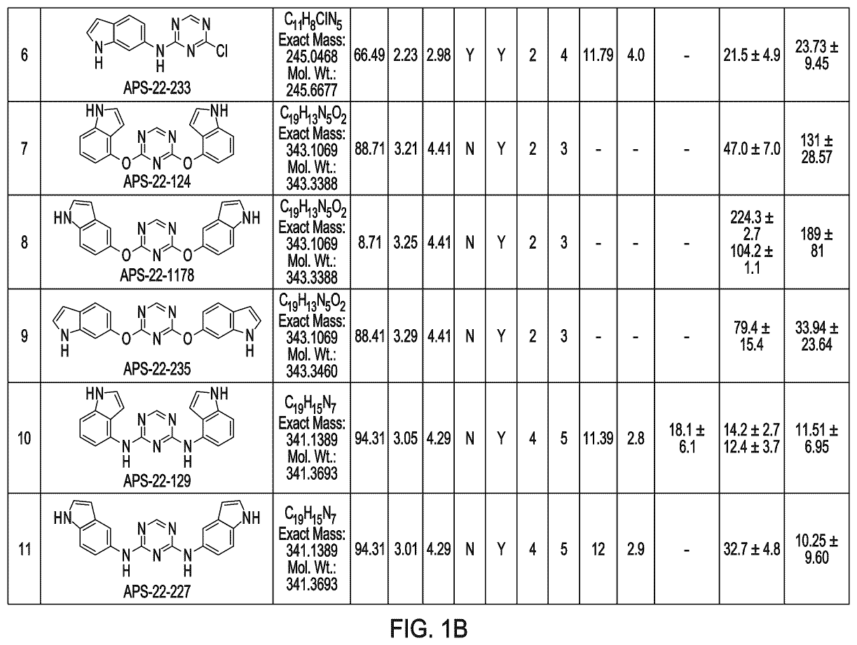

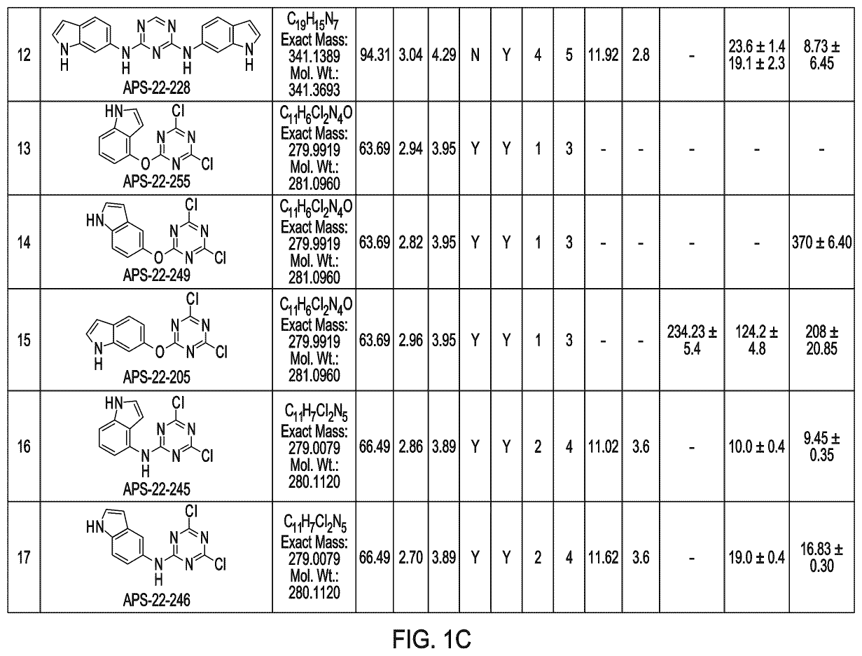

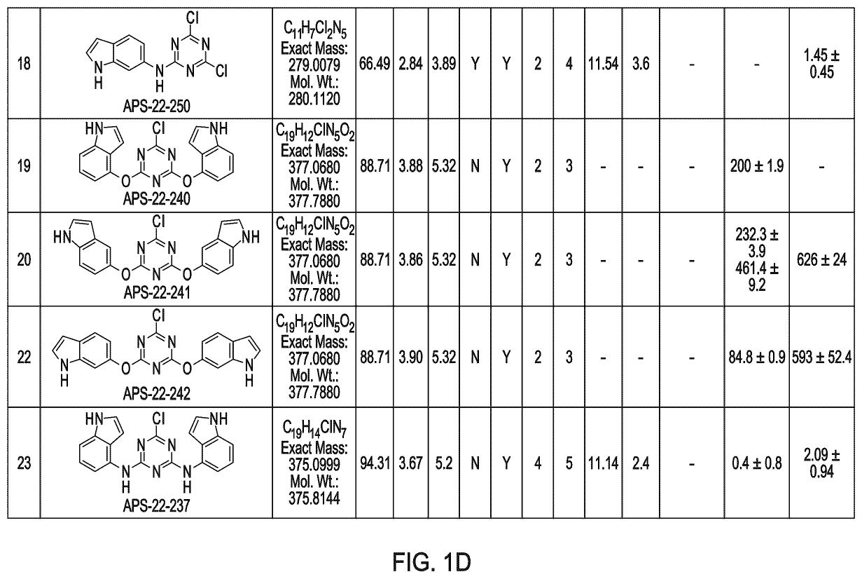

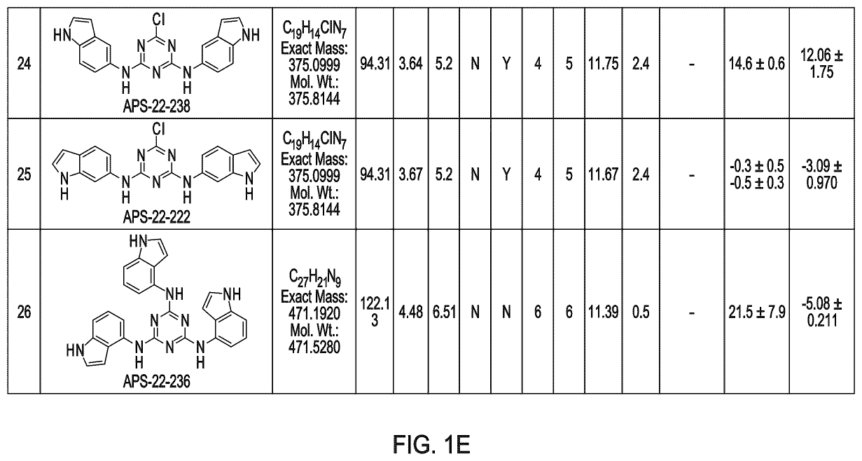

Thioflavin fluorescence assays. Thioflavin fluorescence assays were used to monitor fibril formation of recombinant α-syn, tau isoform 2N4R, treated with control DMSO and different compounds. Kinetics of α-syn fibrillization were performed using thioflavin T (ThT) as published previously.³²′ ³⁷ The fluorescence emission experiments were performed with the excitation and emission wavelengths set at 440 and 485 nm, respectively, with a Synergy HT multi-mode microplate reader (BioTek, Winooski, VT). Samples were measured in three replicates and the experiments were repeated three times using at least two different stock solutions. For each time point, arbitrary units of fluorescence were calculated from the mean values normalized against the maximum value in each completed assay. All results contained in FIG. 1 were presented as mean±SEM.

The kinetics of aggregation of p-tau isoform 1N4R (6 μM) was followed using thioflavin S as published previously. Concerning the tau (isoform 2N4R) kinetics of fibril formation, measurements of ThT fluorescence were performed with a solution of the protein diluted to a final concentration of 6 μM in phosphate-buffered saline (PBS) (pH 7.4) supplemented with 1.5 μM heparin, 20 μM ThT, 2.5 mM DTT, and 100 μM compound. Aliquots of the diluted protein solution (100 μL each) were pipetted into the wells of a 96-well plate, and a Teflon ball was added to each well. The plate was incubated at 37° C. with constant shaking at 1,000 rpm in a Tecan Spark plate reader. ThT fluorescence was measured every 15 min with excitation and emission wavelengths of 440 nm and 480 nm, and the data were plotted using GraphPad Prism.

The fluorescence intensities of the newly synthesized compounds were compared with the control (DMSO) and the reduction of the percentage FI to 15% or less, was considered as an ideal value to move the compound to tier-2 assays. Based on the data obtained for the triazine derivatives 1-25, N-substituted triazine derivatives (compounds 4-6, 10-12, 16-18, 23-26) were significantly more potent than the O-substituted derivatives (compounds 1-3, 7-9, 13-15, 19-22) in inhibiting the aggregation (FIG. 1).

For each compound, the physicochemical parameters were predicted using SwissADME. Blood-Brain Barrier (BBB) Prediction Server (www[dot]cbligand[dot]org/BBB/), and Chemaxon. The chemical structures and physicochemical parameters are indicated in FIG. 1. During the design of these molecules, the following favorable physicochemical properties to cross the BBB were considered based on the Pfizer study: a MW≤397.9, a calculated partition coefficient (c log P) ranging from 1.7 and 3 (below ~2.8), and a topological polar surface area (TPSA) below 80-90 Å². Based on that study, a Central Nervous System Multiparameter Optimization scoring (CNS MPO candidates) was obtained for each compound. All compounds have a molecular weight below 400. Compounds 1 to 6 have a c log P value below 2.8. Compounds 1 to 9 have a TPSA below 80-90 Å². On a scale of 6, the CNS MPO scores ranging from 3.5 to 4.5 are considered having potential to cross the BBB. Only compounds 4 to 6 have a CNS MPO within this range~4.0. Compounds 4 to 6 meet all desired parameters. From observations in FIG. 1, the monosubstituted triazine linkers exhibit more favorable scores to cross the BBB.

FIG. 1. Chemical structure, physicochemical descriptors, and the effect of compounds on alpha-synuclein (α-syn) aggregation measured as fluorescence intensity using ThT assay. TPSA, Log P, and Log D were obtained with SwissADME. BBB and pKa were obtained from the Blood-Brain Barrier Prediction Server (https://www.cbligand.org/BBB/) and Chemaxon, respectively. CNS MPO scores were assessed as published previously, The ThT (%) represent the maximum fluorescence intensity obtained at the plateau phase of the kinetics of fibrillization using 6.25 μM of α-syn. Compounds were tested at 100 μM (molar ratio 1:16). BBB, blood-brain barrier; C log D, calculated distribution coefficient at 7.4; C log P, calculated partition coefficient; CNS MPO, Central Nervous System Multi Parameter Optimization; HBA, Hydrogen Bond Acceptor; HBD, Hydrogen Bond Donor; LR5, Lipinski's rule of five; pKa, negative log of acid dissociation constant; ThT, Thioflavin T; TPSA, Topological polar surface area.

Specifically, N-substituted triazine derivatives namely, compounds 18, 22 and 24 exhibited the highest potency with FI values of 1.45±0.45%, 2.09±0.94%, and −0.3±0.5% respectively. In case of monosubstituted derivatives, the substitution of H (compounds 1-3) with Cl at the 4^{th} position of O-triazines (compounds 13-15) led to the loss of activity, but in case of N-triazines (compounds 4-6 and 16-18) there was a significant improvement in FI reduction with 6-aminoindolyl group (compound 18) being the most preferred substituent. Interestingly in case of di-substituted derivatives, the substitution of H (compounds 7-9) with Cl at the 4^{th} position of O-triazines (compounds 19-21) gave similar results when compared with the mono-substituted derivatives i.e., the loss of activity. As expected, the di-substituted N-triazines with Cl on the 4^{th} position of triazine (compounds 22-24) showed high potency with FI in the range of −0.3-12%, with 6-aminoindolyl group (compound 24) being the most preferred substituent. The effect of substituting 4-aminoindole at the three positions (2,4,6) of triazine ring was also investigated and the FI measured for compound 25 was found to be 21.5±7.9%.

Compound 10, a triazine derivative, has the highest effect on the kinetics of α-syn fibril formation as monitored by thioflavin-T (THT) fluorescence assay. The impact of different substitutions on the triazine ring on the kinetics of α-syn fibril formation was assessed using thioflavin T (ThT) assays by measuring the percentage fluorescence intensities (FI). The FIs of the newly synthesized compounds were compared with the control (DMSO), and the reduction of the percentage FI to 15% or less was considered as cut off value to move the compound to orthogonal (i.e., transmission electron microscopy) and tier-2 assays (e.g., cell-based assays). Based on the data obtained for the triazine derivatives 1-12, N-substituted triazine derivatives (compounds 4-6, 10-12) were greater inhibitors of α-syn aggregation than the O-substituted derivatives (compounds 1-3, 7-9) in inhibiting the aggregation (FIG. 1). Specifically, N-substituted triazine derivative namely, compound 10 exhibited the highest anti-aggregation effect with FI values of 13.5±1.7%.

### Example 3 Confirmation of Anti-Fibrillary Activity Using Tau Isoform 2N4R Fibril and ThT Assay

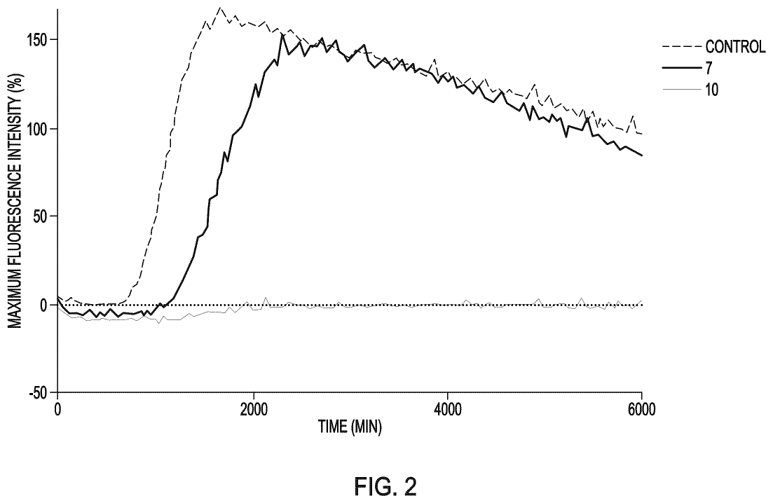

The α-syn kinetics of aggregation following the best N-substituted triazine (compound 10) and 0-substituted counterpart (compound 7, negative control) are shown in FIG. 2). Compound 10 delayed slightly the lag time required for the fibril elongation.

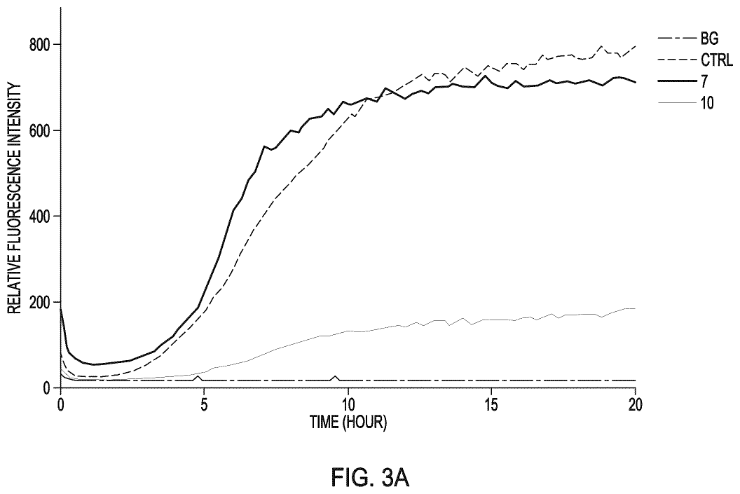

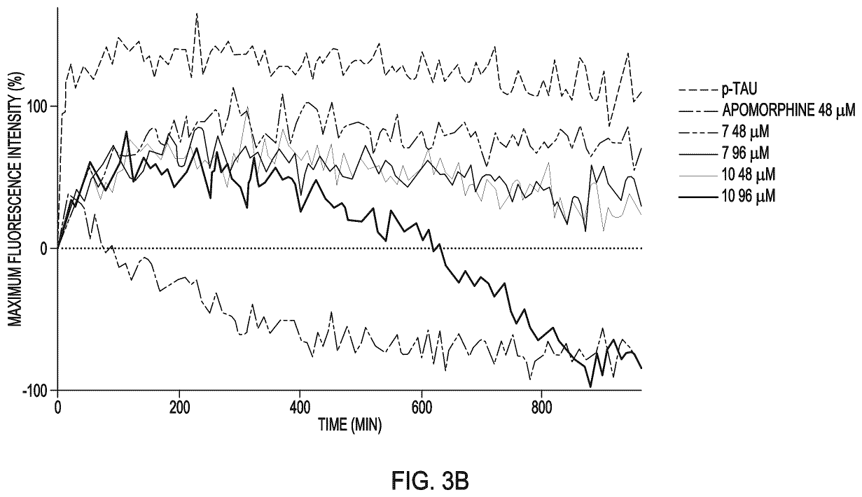

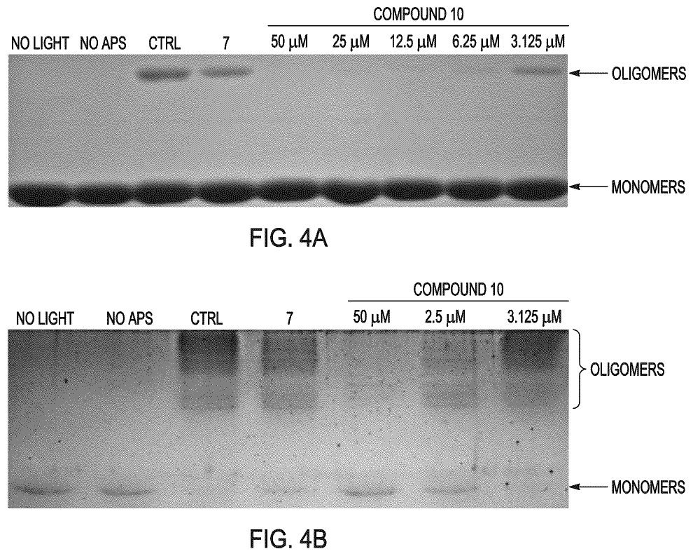

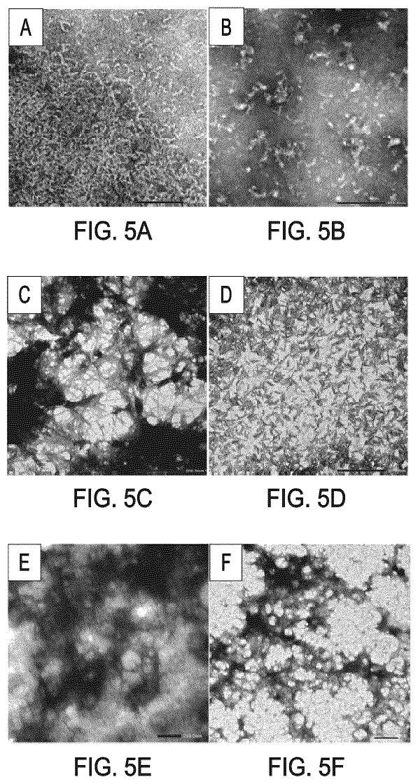

Compound 10 exhibits an anti-fibrillary activity on tau isoform 2N4R and phosphorylated tau (p-tau) isoform 1N4R. The next set of experiments was aimed at determining how well the compound that exhibited the best anti-fibrillar effects on α-syn fibrillization also affected the rate of tau fibrillization. Based on α-syn ThT experiments (FIG. 1), the best triazine compound 10 was selected and tested for its effect on the fibrillization of tau isoform 2N4R using a ThT fluorescence assay. Consistent with the α-syn ThT results, the pre-selected triazine compound 10 markedly inhibited tau isoform 2N4R fibrillization and negative control (compound 7) failed to reduce tau fibril formation (FIG. 3A). Compound 10 showed a slower elongation rate, as evidenced by the slope of the fluorescence versus time curve following the lag phase, in addition to reduced fibrillization levels, as reflected by reduced ThT fluorescence intensity.

The anti-fibrillary effect of compound 10 was challenged using 6 μM of hyperphosphorylated tau (p-tau) isoform 1N4R (FIG. 3B). Apomorphine is a known powerful inhibitor of p-tau fibril formation³¹ and reduction of fibril formation was observed at 48 μM. Compound 10 reduced the fibril formation overtime, at high concentration 98 μM, reaching the apomorphine fluorescence intensity at the end of the kinetics (FIG. 3B). A lower concentration of compound 10 (48 μM) and compound 7 (at both 48 and 98 μM), a negative control, did not reduce the fluorescence intensity at the same level than apomorphine.

### Example 4 Monitoring α-Syn and Tau Oligomer Formation with Potent Triazine Derivatives by Photo-Inducing Cross-Uncoupled Protein (PICUP) Assay

PICUP is a technique in which oligomer formation is induced by cross-linking. To induce oligomerization by cross-linking, α-syn (from Rpeptide, LLC) and tau isoform 2N4R were diluted in 10 mM phosphate buffer (pH 7.4) to reach a final concentration of 10 μM. Different compounds were added to the protein solution at a final concentration of 50 μM, resulting in a molar ratio of 1:5. To confirm the gradual effect of compounds on the inhibition of α-syn oligomerization, compounds were tested at final concentration of 3.125, 6.25, 12.5, 25, and 50 μM. The controls consisted of samples without light exposition, without Ru(bpy) or ammonium persulfate, and without compound (i.e., 0.125% DMSO). The cross-linking reaction was initiated by the addition of 2 μL of Ru(bpy) (300 μM final concentration) and 2 μL ammonium persulfate (6 mM final concentration).³² Samples were irradiated immediately. Light exposure was of a 1 second duration for α-syn and a 3 second duration for tau isoform 2N4R, with a 53 W (120 V) incandescent lamp installed in a homemade dark-box. Each tube contained a final volume of 20 μL. After irradiation, 8.3 μL of Laemmli loading buffer containing 15% 0-mercaptoethanol was immediately added to the solution, followed by incubation at 95° C. for 10 min. The cross-linked samples were separated on a 16% SDS-PAGE gel and visualized by Coomassie blue staining.

High molecular weight oligomers were detected around 50 kDa for α-syn and 190 kDa for tau in the Coomassie blue-stained 16% polyacrylamide gel. Compound 7 was used as a negative control due to its nonexistent anti-fibrillar activity. As expected, compound 7 did not reduce the formation of α-syn and tau oligomers (FIG. 4). Compound 10 inhibited both α-syn and tau oligomer formation. For this reason, different concentrations were tested to confirm the anti-oligomer activity. Compound 10 was effective in reducing oligomerization of α-syn and tau isoform 2N4R in a dose-dependent manner (FIG. 4). Because of the dual effect observed on these two prone-to-aggregate proteins, it was decided to proceed with transmission electron microscopy (TEM) as orthogonal assays to confirm the effect on α-syn, tau isoform 2N4R, and p-tau fibril (1N4R) formation.

### Example 5 TEM Analysis on α-Syn, Tau Isoform 2N4R and p-Tau Isoform 1N4R Pre-Treated with Best Triazine Compounds

To confirm the general anti-fibrillar effect of compound 10, transmission electron microscopy (TEM) was used as an orthogonal assay. After incubating recombinant proteins (at 60 μM) with 600 μM compound 10 for 24 hours at 37° C., TEM was utilized to detect fibril formation.³² A volume of 10 μL was applied on a 400-mesh Formvar-carbon-coated copper grid (Electron Microscopy Sciences, Hatfield, PA). The grids were incubated for 1 minute and washed three times with distilled water. They were carefully air-dried and incubated for 1 minute in a fresh solution of 1% uranyl acetate. Samples were air-dried and observed using a transmission microscope. Visualization of the grids was performed with transmission electron microscopy (JEOL 1400 Flash, Japan). Acquisition of pictures was performed with the following settings: accelerating voltage of 100 kV and magnification of 40k.

Proteins were tested at 60 μM to visualize enough fibrils and to compare the effects of compound 10 with DMSO (FIG. 5). Molar ratio protein and compound used was 1:10. Compound 10 reduced fibril formation for each treated recombinant protein (α-syn, tau, p-tau) in comparison with controls (1.5% DMSO). For α-syn, treatment with compound 10 resulted in a range of shorter fibrils to amorphous protein. Application of tau isoform 2N4R resulted in very dense mat of fibrils after incubation of 24 h at 37° C. By contrast, needle-shaped short fibrils were observed with tau isoform 2N4R incubated with compound 10. Less ultrastructural changes were observed with p-tau subjected to compound 10 (justifying acquisition of pictures at 20K). However, less fibrils resulted from this treatment.

Key results: α-syn: Control: thin intertwined fibrils. Compound 7: packets of fine fibrils with a few spherical structures encountered on the grid. Compound 10 and 25: rare short fibrils. Compound 23: quasi absence of fibrils, debris. Tau 2N4R: Control: very dense mats of fibrils. Compound 7: fibrils surrounded by debris admixed with disrupted fibrils. Compound 10: very short needle shaped fibrils admixed with debris. Compound 23: very rare fibrils encountered on the grid, debris admixed with amorphous materials. Compound 25: medium size needle-shaped fibrils.

### Example 6 Biological Evaluation of the Effect of Triazine Compounds on α-Syn Inclusion in Neuroblastoma Cells

The dox-inducible neuroblastoma cell line M17D-TR/αS3K::YFP was used to evaluate the effect of compounds in preventing the formation of inclusion following induction with Dox. Dox-inducible neuroblastoma cells M17D-TR/αS-3K::YFP have been used previously. 96-well plates were used with cellular density of 30,000 cells per well. Compounds were added after 24 h and aS-3K::YFP transgene expression was induced 48 h later. Induction was done by adding 1 μg per mL (final concentration) dox to culture media. Cells were incubated in the Incucyte Zoom 2000 platform (Essen Biosciences) and images (green, bright field) were taken continuously. Endpoint analysis of inclusion formation or growth was performed 48 h after induction (96 h after plating). The Incucyte processing definition ‘Inclusions’ was created as follows: Parameters, Fixed Threshold, Threshold (GCU) 50; Edge Split On, Edge Sensitivity 100; Cleanup, Hole Fill (μm²): 10, Adjust Size (pixels): 0; Filters, Area (μm²): max 50, Mean Intensity: min 60, Integrated Intensity: min 2000. Cell confluence was measured by the processing definition ‘Cells’: Parameters, Segmentation Adjustment 0.7; Cleanup, all parameters set to 0; Filters, Area (μm²): min 345.00. As described previously for the evaluation of protein expression by SDS-PAGE and Western Blotting in the LiCor system, αS-specific monoclonal antibody 4B12 (Thermofisher, Waltham, MA; 1:1000) and a polyclonal antibody to GAPDH (Sigma-Aldrich, St. Louis, MO, G9545; 1:5000) were used.

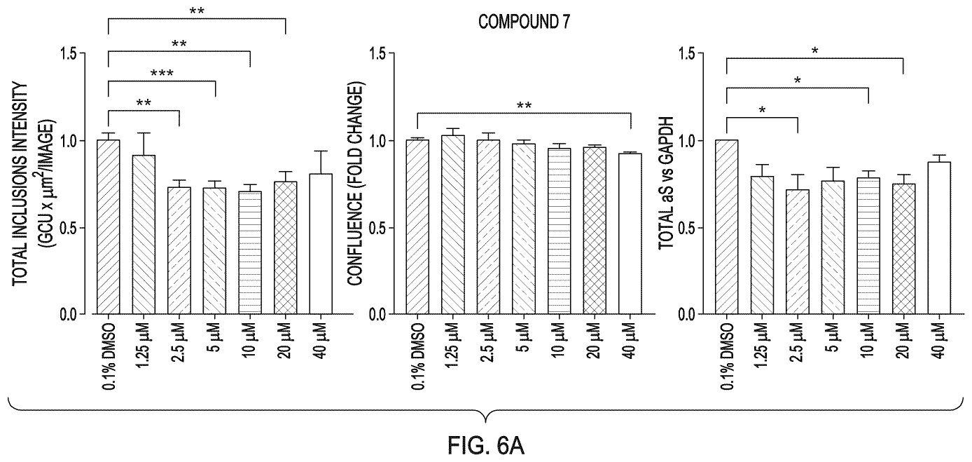

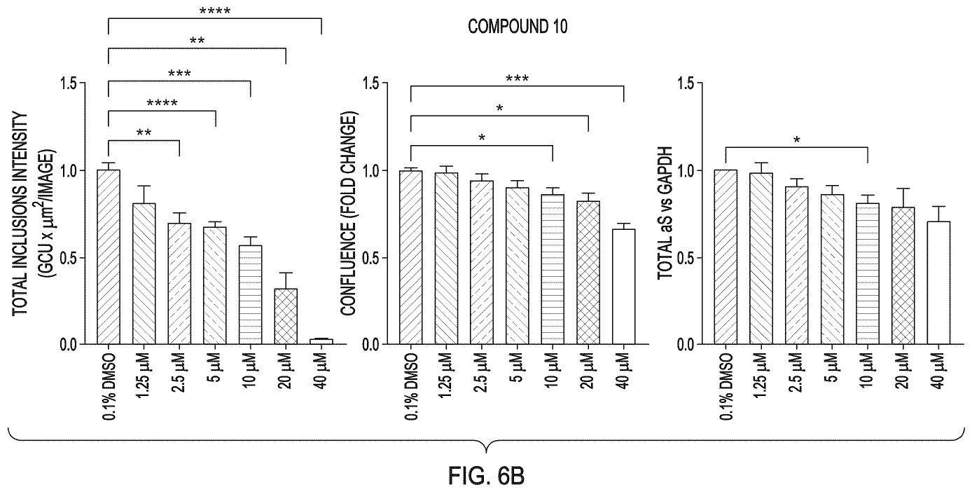

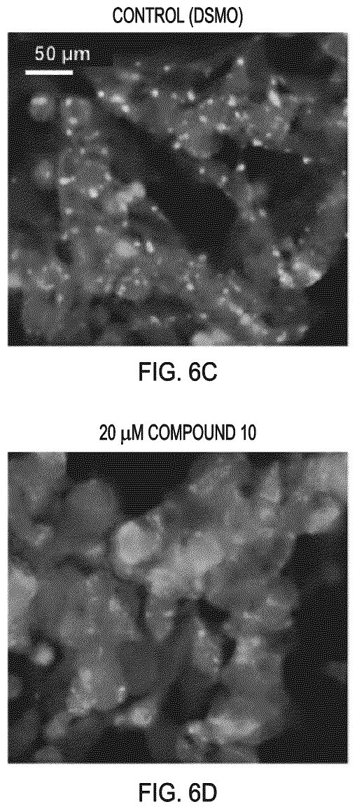

Compound 7 demonstrated a weak effect on fibril formation and no effect on oligomerization. As expected, very minimal changes in the numbers of inclusion were observed when cells were subjected to compound 7 treatments at various concentrations from 2.5 to 20 μM (FIG. 6). By contrast, compound 10 reduced α-syn inclusion formation at concentrations ranging from 2.5 to 40 μM. The most significant decreases occurred at 20 and 40 μM. Compound 10 demonstrated certain small level of toxicity starting at 10 μM but more intense toxicity at 40 μM. α-syn expression levels were reduced with both compounds at a concentration of 10 μM, in addition to other concentration for compound 7, i.e., 2.5 and 20 μM.

Key results: Compound 7 demonstrated a weak effect on the fibril formation and no effect on the oligomerization. Very minimal changes in the numbers of inclusion were observed when cells were subjected to compound 7 treatments at 2.5 to 20 μM. Compound 10 reduced inclusion formation at concentrations ranging from 2.5 to 40 μM. Most significant decrease was observed at 20 and 40 μM. Compound 10 demonstrated a certain level of toxicity starting at 10 M but considerable at 40 μM. α-syn expression levels were reduced with both compounds at a concentration of 10 μM, in addition to other concentrations for compound 7, i.e., 2.5 and 20 μM.

FIG. 6 shows compound 10 reduced inclusion formation in a dose-dependent manner using the M17D neuroblastoma cells that express inclusion-prone α-Synuclein 3K::YFP. A) M17D cells that express an αS3K::YFP fusion protein (dox-inducible) were treated with 0.1% DMSO (control) and compounds 7 (negative control) at various concentrations at t=24 hrs. Cells were induced with dox at t=48 hours. Punctate YFP signals were measured and normalized to 0.1% DMSO at t=96 hours. The cell confluence percentage was plotted. For the first two graphs, four independent experiments (N=4; n=24; except DMSO, n=12) were performed. Total aS/calnexin ratios were plotted following WB analyses, normalized to 0.1% DMSO (N=4; n=24, except DMSO, n=4). Data are presented as fold-change relative to 0.1% DMSO. Ordinary one-way ANOVA plus Dunnett's multiple comparison test (*, p<0.1, **, p<0.01, ***, p<0.001, ****, p<0.0001). B) Same as panel A, but with compounds 10.

### Example 7 Compound Disaggregates Aβ-Plaques and Paired Helical Filaments Ex Vivo

The disaggregation effect of compound 10 was tested using Aβ-plaques and paired helical filaments isolated from AD brains. This alleviates concerns in using recombinant proteins for drug candidate screening to evaluate potential anti-aggregation activity using endogenous aggregates from diseased brain. Prior studies demonstrated differences in the ultrastructure of fibrils isolated from AD brains or generated from recombinant proteins.

Amyloid-β plaques were purified from postmortem human AD brains obtained from patients diagnosed with advanced Alzheimer's (Braak stage V or VI) (Tissue Biobank at Case Western Reserve University-Cleveland Clinic) using ultracentrifugation with a sucrose gradient and cell sorting, as previously published.³⁸⁻³⁹ Purified amyloid-β plaques were examined with polarized and fluorescence microscopy (Congo Red and Thioflavin-S staining) and scanning electron microscopy. Each aliquot of amyloid-β plaques was solubilized with 60 μL of 10 mM PBS buffer (pH 7.4). One extraction consisted of 0.28 mg/ml of protein, as measured with nanodrop (A260/280 nm). A volume of 20 μL of extraction was incubated with 1.5% DMSO (control) or compound 10 (50 μM) for 120 hours at 37° C. prior to visualization by transmission electron microscopy.

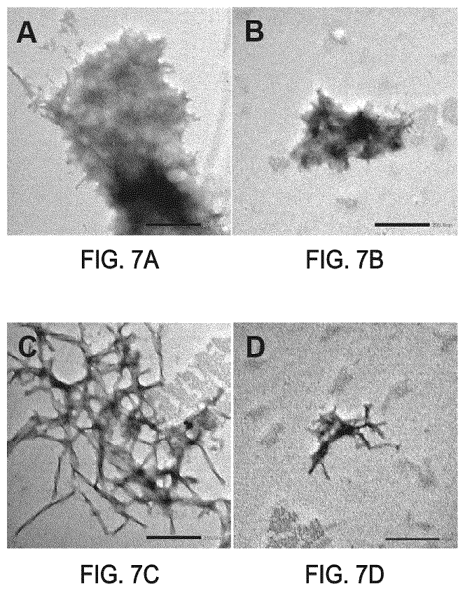

The protein quantification of the plaques isolated from AD brains were measured with nanodrop. 3 replicates resulted in a concentration of 0.27 mg/ml of proteins. Compound 10 was tested at 50 μM based on the protein levels and results obtained with PICUP experiments (FIG. 7). AP-plaque samples were incubated with 1.5% DMSO (control) or compound 10 (50 μM) in 10 mM PBS buffer (pH 7.4) for 120 hours at 37° C. prior to visualization by transmission electron microscopy. During the visualization of the grids, several paired helical filaments were observed. In contrast to the control (1.5% DMSO), disaggregation experiments showed smaller AP-plaques and shorter paired helical filaments after incubation of 5 days at 37° C. with compound 10 (FIG. 7).

Symmetric triazine derivatives were synthesized to evaluate the anti-aggregation effects of mono- and di-substitutions and the presence of aminoindole or hydroxyindole moieties on α-syn, tau isoform 2N4R, and p-tau isoform 1N4R. Through analysis of α-syn ThT fluorescence, it was demonstrated that N-substituted triazines are more efficacious inhibitors of fibril formation in contrast to O-substituted triazine derivatives. The best compound, compound 10, was tested on tau isoform 2N4R for the evaluation of anti-oligomer and anti-fibrillar activities. Compound 10 was the most effective inhibitor of α-syn and tau 2N4R oligomerization and was capable of preventing the formation of oligomers in a dose-dependent manner. Compound 10 is a symmetrically di-substituted 4-aminoindole triazine derivative with some favorable physicochemical properties to cross the blood brain barrier. Moreover, compound 10 reduced α-syn inclusion body formation in the dox-inducible neuroblastoma cell line M17D-TR/αS3K::YFP starting at 2.5 μM and disaggregated human Aβ plaques and NFTs at 50 μM.

Based on the data obtained from the biophysical and biological assays, compound 10 seems to be the most promising triazine derivative in preventing fibril formation, oligomerization, and inclusion formation of prone-to-aggregate proteins.

### Example 8

Compound 10 has a short half-life and data demonstrated that the compound tested crossed the blood brain barrier using CD1 mice. The concentration ration plasma:brain is near to one which is adequate. The short half-life could be attributed to small administration of compound (1 mg/kg), elimination, or CYP P450 metabolism.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure. The term “or” is used to refer to a nonexclusive “or” unless otherwise indicated.

Unless defined otherwise, all technical and scientific terms used herein have the same meaning as commonly understood to one of ordinary skill in the art. The following terms and phrases shall have the meaning indicated.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value, such as within 10%, within 5%, or within 1% of a stated value or stated limit of a range) provided that one of ordinary skill in the art would consider equivalent to the recited value (e.g., having the same function or result). The term “substantially” can allow for a degree of variability in a value or range, for example, within 90%, within 95%, 99%, 99.5%, 99.9%, 99.99%, or at least about 99.999% or more of a stated value or of a stated limit of a range.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

1. A compound of the formula: 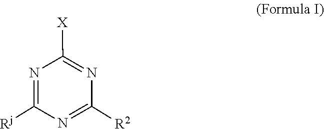
or a pharmaceutically acceptable salt thereof;
wherein R¹ is 4-aminoindole, 5-aminoindole, 6-aminoindole, or 7-aminoindole; R² is 4-aminoindole, 5-aminoindole, 6-aminoindole, or 7-aminoindole; and X is H, halo, NO₂, NH₂, OH, or O-alkyl.

2. The compound of claim 1, wherein halo is Cl, F, I or Br.

3. The compound of claim 1, wherein R¹ and R² are 4-aminoindole, 5-aminoindole, 6-aminoindole, or 7-aminoindole and X is H, halo, NO₂, NH₂, OH, or O-alkyl.

4. (canceled)

5. A compound of the formula: 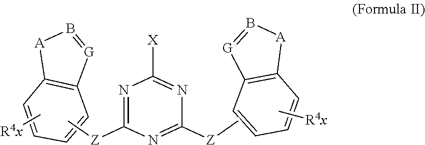
or a pharmaceutically acceptable salt thereof;
wherein:
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl:
each Z is independently O or NR³, wherein R is H or alkyl;
each A is, independently, O, S or NR³, wherein R³ is H or alkyl;
each B is independently N or CR⁵, wherein R⁵ is H, acyl, amido, or alkyl; and
each G is independently N or CR⁵, wherein R⁵ is —I, acyl, amido, or alkyl.

6. (canceled)

7. The compound of claim 5, wherein each Z is independently O or NH or each A is independently NR³, wherein R³ is optionally H or C₁-C₆ alkyl.

8. (canceled)

9. (canceled)

10. (canceled)

11. (canceled)

12. (canceled)

13. (canceled)

14. The compound of claim 5, wherein each A is O or each A is S.

15. (canceled)

16. (canceled)

17. (canceled)

18. The compound of 5, wherein each B is independently CR⁵, wherein each CR⁵ is optionally CH; or each B is N.

19. (canceled)

20. (canceled)

21. (canceled)

22. (canceled)

23. (canceled)

24. The compound of claim 5, wherein each G is independently CR⁵, wherein each CR⁵ is optionally CH; or each B is N.

25. (canceled)

26. (canceled)

27. (canceled)

28. (canceled)

29. (canceled)

30. The compound of claim 5, wherein each R⁴ is H or X is H.

31. (canceled)

32. (canceled)

33. (canceled)

34. The compound of claim 5, wherein the compound is of the formula: 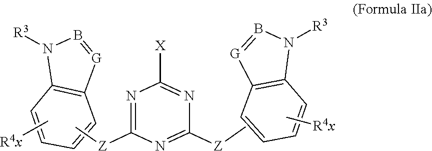
or a pharmaceutically acceptable salt thereof;
wherein:
each R³ is independently I or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl;
each Z is independently O or NR³, wherein R³ is H or alkyl;
each B is independently N or CR⁵, wherein R⁵ is H, acyl, amido, or alkyl; and
each G is independently N or CR⁵, wherein R⁵ is H, acyl, amido, or alkyl.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The compound of claim 5, wherein the compound is of the formula: 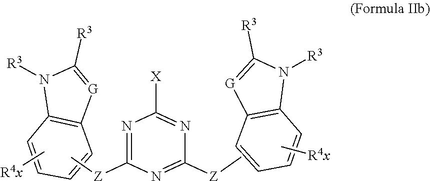
or a pharmaceutically acceptable salt thereof;
wherein:
wherein each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl,
each Z is independently O or NR³, wherein R³ is H or alkyl; and
each G is independently N or CR⁵, wherein R⁴ is H, acyl, amido, or alkyl.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. The compound of claim 5, wherein the compound is of the formula: 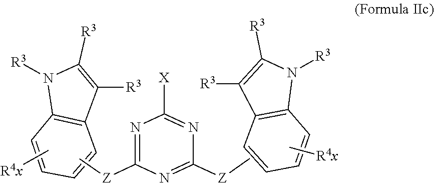
or a pharmaceutically acceptable salt thereof;
wherein:
each R³ is independently H or alkyl;
each x is independently 1, 2, or 3;
each R⁴ is independently H, halo, nitro, cyano, alkoxy, amino or two R⁴ groups and the carbon atoms to which they are attached can form an aryl or a heterocyclyl group;
X is H, halo, NO₂, NH₂, OH, or O-alkyl; and
each Z is independently O or NR³, wherein R³ is H or alkyl.

75. (canceled)

76. (canceled)

77. (canceled)

78. (canceled)

79. (canceled)

80. (canceled)

81. (canceled)

82. (canceled)

83. (canceled)

84. (canceled)

85. The compound of claim 5, wherein each group of the formula: 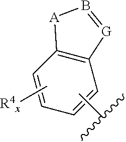
is independently a group of the formula: 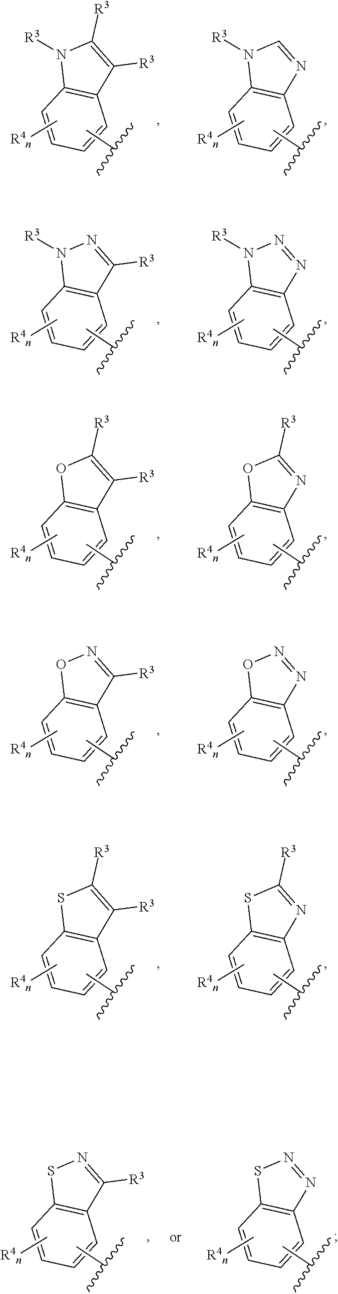
wherein:
each R³ is independently H or alkyl;
each x is independently 1, 2, or 3; and
each R⁴ is independently H, halo, nitro, cyano, alkoxy, amino or two R⁴ groups and the carbon atoms to which they are attached can form an aryl or a heterocyclyl group.

86. (canceled)

87. The compound of claim 85, wherein each R³ is H or C₁-C₆ alkyl.

88. (canceled)

89. The compound of claim 85, wherein each R⁴ is independently H.

90. (canceled)

91. (canceled)

92. A pharmaceutical composition comprising the compound of claim 1 and a pharmaceutically acceptable carrier.

93. A method of inhibiting tubulin-associated unit (tau) and/or alpha-synuclein (α-syn) protein aggregation in a subject having, or at risk for, tau and/or α-syn protein aggregation, which method comprises administering to the subject the composition of claim 92 in an amount effective to inhibit tau protein and/or α-syn protein aggregation, whereupon tau and/or α-syn protein aggregation is inhibited in the subject having, or at risk for tau and/or α-syn protein aggregation.

94. The method of claim 93, wherein tau is tau isoform 2N4R or phosphorylated tau isoform 1N4R.

95. (canceled)

96. The method of claim 93, wherein the subject has, or is at risk for, Alzheimer's disease or the subject has, or is at risk for, Parkinson's disease.

97. (canceled)

98. The method of claim 93, wherein the subject has neuroblastoma and the formation of α-syn inclusions is inhibited.
